(19) United States
(12) Patent Application Publication
(10) Pub. No.: US 2025/0231177 A1
(43) Pub. Date: Jul. 17, 2025

(54) METHOD OF CHARACTERIZING TUMORS

(71) Applicant: University of Florida Research Foundation, Inc., Gainesville, FL (US)

(72) Inventors: Elias Sayour, Newberry, FL (US); Hector Ruben Mendez-Gomez, Gainesville, FL (US); Hilary Geffrard, Gainesville, FL (US); Sadeem Qdaisat, Gainesville, FL (US); Wallace Gregory Sawyer, Gainesville, FL (US)

(21) Appl. No.: 18/854,015

(22) PCT Filed: Apr. 3, 2023
(86) PCT No.: PCT/US23/17281

§ 371 Date: Oct. 3, 2024

Related U.S. Application Data
(60) Provisional application No. 63/345,680, filed on May 25, 2022.
(60) Provisional application No. 63/326,941, filed on Apr. 4, 2022.

(51) Int. Cl. G01N 33/50; A61P 35/00; C07K 16/28; C12N 5/09; G01N 33/68

(52) U.S. Cl. CPC G01N 33/5023; A61P 35/00; C07K 16/2827; C12N 5/0693; G01N 33/6866; G01N 33/6869; G01N 2333/521; G01N 2333/5412; G01N 2333/56; G01N 2800/52

(57) ABSTRACT

The present disclosure provides a method of identifying a tumor for immunotherapy. The method comprises culturing tumor cells obtained from a subject; exposing the tumor cells to nanoparticles comprising a positively-charged surface and an interior comprising (i) a core and (ii) at least two nucleic acid layers, wherein each nucleic acid layer is positioned between a cationic lipid bilayer; and measuring interferon-alpha and interleukin 6 (and optionally Chemokine (C-C motif) ligands 4) produced by the tumor cells. A method of treating a subject with cancer also is provided. The method comprises culturing tumor cells from the subject; exposing the tumor cells to the nanoparticles; measuring interferon-alpha and interleukin 6 (and optionally Chemokine (C-C motif) ligands 4) produced by the tumor cells; and administering an immune checkpoint inhibitor to the subject. Any of the methods may also comprise measuring CXCL 10.

### CROSS REFERENCE TO RELATED APPLICATIONS AND INCORPORATION BY REFERENCE

This application claims the benefit of U.S. Provisional Patent Application No. 63/326,941, filed Apr. 4, 2022, and U.S. Provisional Patent Application No. 63/345,680, filed May 25, 2022, each of which is hereby incorporated by reference in their entireties.

The following applications also are incorporated by reference: International Patent Application No. PCT/US20/42606, filed Jul. 17, 2020; International Patent Application No. PCT/US21/16925, filed Feb. 5, 2021; and International Patent Application No. PCT/US21/18831, filed Feb. 19, 2021.

### GRANT FUNDING DISCLOSURE

This invention was made with government support under grant number R37 CA251978, awarded by the National Institutes of Health. The government has certain rights in the invention.

### FIELD OF THE INVENTION

This application relates to methods of characterizing tumors with respect to the ability to respond to immunotherapy.

### BACKGROUND

Cancer immunotherapy relies on the cytotoxic potential of activated T cells, which scavenge to recognize and reject tumor associated or specific antigens (TAAs or TSAs). Unlike most drug agents, activated T cells can traverse the blood brain barrier (BBB) via integrin (i.e., LFA-1, VLA-4) binding of ICAMs/VCAMs. T cells can be ex vivo activated in co-culture with dendritic cells (DCs) presenting TAAs/TSAs or through transduction with a chimeric antigen receptor (CAR). Alternatively, T cells can be endogenously activated using cancer vaccines. Significant challenges in realizing the full potential of immunotherapy remain. In a randomized phase III trial for patients with primary GBM, peptide vaccines targeting the tumor specific EGFRVIII surface antigen failed to mediate enhanced survival benefits over control vaccines. Cancer immunotherapy with immune checkpoint inhibitors (ICIs) has shown significant promise against malignancies with immunologically active (“hot”) microenvironments, however, this therapy has failed in clinical trials for patients with immunologically inactive (“cold”) tumors.

### SUMMARY

The present disclosure provides a method of identifying a tumor for immunotherapy or characterizing a tumor with respect to its ability to respond to immunotherapy. The method comprises culturing tumor cells obtained from a subject; exposing the tumor cells to nanoparticles comprising a positively-charged surface and an interior comprising (i) a core and (ii) at least two nucleic acid layers, wherein each nucleic acid layer is positioned between a cationic lipid bilayer; and measuring a type 1 interferon (e.g., interferon-alpha and/or interferon-beta), C-X-C Motif Chemokine Ligand 10 (CXCL10), and/or interleukin 6 (IL-6) produced by the tumor cells. In various aspects, the method comprises measuring interferon-alpha and CXCL10 produced by the tumor cells, and optionally further comprising measuring interferon-beta and/or IL-6. The disclosure further provides a method of identifying a tumor for immunotherapy or characterizing a tumor with respect to its ability to respond to immunotherapy, wherein the method comprises culturing tumor cells obtained from a subject; exposing the tumor cells to nanoparticles comprising a positively-charged surface and an interior comprising (i) a core and (ii) at least two nucleic acid layers, wherein each nucleic acid layer is positioned between a cationic lipid bilayer; and measuring interferon-alpha, Chemokine (C-C motif) ligands 4 (CCL4), and/or interleukin 6 (IL-6) produced by the tumor cells. In various aspects, the method comprises measuring interferon-alpha and IL-6 produced by the tumor cells, and optionally further comprises measuring CCL4 (e.g., the method comprises measuring all three of interferon-alpha, CCL4, and IL-6 produced by the tumor cells). The method may further comprise measuring CXCL10. In exemplary aspects, the nanoparticle comprises at least four or five or more nucleic acid layers, each of which is positioned between a cationic lipid bilayer. In various aspects, the outermost layer of the nanoparticle comprises a cationic lipid bilayer. In various instances, the surface comprises a plurality of hydrophilic moieties of the cationic lipid of the cationic lipid bilayer. In exemplary aspects, the core comprises a cationic lipid bilayer. Optionally, the core comprises less than about 0.5 wt % nucleic acid. In exemplary instances, the nanoparticle is characterized by a zeta potential of about +40 mV to about +60 mV, optionally, about +45 mV to about +55 mV. The nanoparticle in various instances, has a zeta potential of about 50 mV. In some aspects, the nucleic acid molecules are present at a nucleic acid molecule:cationic lipid ratio of about 1 to about 5 to about 1 to about 20, optionally, about 1 to about 15, about 1 to about 10 or about 1 to about 7.5. In various aspects, the nucleic acid molecules are RNA molecules, optionally, messenger RNA (mRNA). Optionally, the method further comprises administering the nanoparticles to the subject. Also optionally, the method further comprises administering an immune checkpoint inhibitor (ICI) to the subject and/or a population of second nanoparticles comprising a positively-charged surface and an interior comprising (i) a core and (ii) at least two nucleic acid layers, wherein each nucleic acid layer is positioned between a cationic lipid bilayer.

A method of treating a subject with cancer also is provided. The method comprises culturing tumor cells obtained from the subject; exposing the tumor cells to nanoparticles comprising a positively-charged surface and an interior comprising (i) a core and (ii) at least two nucleic acid layers, wherein each nucleic acid layer is positioned between a cationic lipid bilayer; measuring interferon-alpha (or another type 1 interferon, such as interferon-beta), CCL4, and/or IL-6 produced by the tumor cells; and administering an immune checkpoint inhibitor to the subject. In various aspects, the method comprises measuring interferon-alpha and IL-6 produced by the tumor cells, and optionally further comprises measuring CCL4 (e.g., the method comprises measuring all three of interferon-alpha, CCL4, and IL-6 produced by the tumor cells). Alternatively, the method comprises culturing tumor cells obtained from the subject; exposing the tumor cells to nanoparticles comprising a positively-charged surface and an interior comprising (i) a core and (ii) at least two nucleic acid layers, wherein each nucleic acid layer is positioned between a cationic lipid bilayer; measuring interferon-alpha, CXCL10, interferon-beta, and/or IL-6 produced by the tumor cells; and administering an immune checkpoint inhibitor to the subject.

Additional features and variations of the materials and methods of the disclosure will be apparent to those skilled in the art from the entirety of this application, including the figures and detailed description, and all such features are intended as aspects of the invention. Features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1A is a series of illustrations of a lipid bilayer, liposome and a general scheme leading to multilamellar (ML) RNA NPs (boxed).

FIG. 1B is a pair of CEM images of uncomplexed NPs (left) and ML RNA NPs (right).

FIG. 2A is an illustration of a general scheme leading to cationic RNA lipoplexes.

FIG. 2B is an illustration of a general scheme leading to cationic RNA lipoplexes.

FIG. 2C is a CEM image of uncomplexed NPs, FIG. 2D is a CEM image of RNA LPXs, and FIG. 2E is a CEM image of ML RNA NPs.

FIG. 2F is a graph of the % CD86+ of CD11c+MHC Class II+ splenocytes present in the spleens of mice treated with ML RNA NPs (ML RNA-NPs), RNA LPXs, anionic LPXs, or of untreated mice.

FIG. 2G is a graph of the % CD44+CD62L+ of CD8+ splenocytes present in the spleens of mice treated with ML RNA NPs (ML RNA-NPs), RNA LPXs, anionic LPXs, or of untreated mice.

FIG. 2H is a graph of the % CD44+CD62L of CD4+ splenocytes present in the spleens of mice treated with ML RNA NPs (ML RNA-NPs), RNA LPXs, anionic LPXs, or of untreated mice.

FIG. 2I is a graph of the % survival of mice treated with ML RNA NPs (ML RNA-NPs), RNA LPXs, anionic LPXs, or of untreated mice.

FIG. 2J is a graph of the amount of IFN-α produced in mice upon treatment with ML RNA NPs (ML RNA-NPs), RNA LPXs, anionic LPXs, or of untreated mice.

FIG. 3 is a table listing the top 620 genes that are representative of the slow cycling cell (SCC) transcriptome.

FIGS. 4A-4E: Cytokine, chemokine, and GFP expression in transfected GL-261, K-LUC, B16F0, and B16F10 OVA cells. FIG. 4A is a bar graph illustrating expression of INF-α by GL-261, K-LUC, B16F0, and B16F10 OVA cells transfected with RNA+ nanoparticles, transfected with RNA-nanoparticles, and left untreated. Supernatant was collected after 24 h incubation following treatment. FIG. 4B is a bar graph illustrating expression of INF-β measured in pg/ml. FIG. 4C is a bar graph illustrating IP-10/CXCL10 expression measured in pg/mL. FIG. 4D is a bar graph illustrating interleukin-6 expression in cells transfected with RNA+ nanoparticles, transfected with RNA-nanoparticles, and left untreated. FIG. 4E is bar graph illustrating GFP intensity measured 24 h after transfection via flow cytometry.

FIG. 5A is an illustration of a microfluidic system for horizontal perfusion of tumoroids with in situ confocal microscopy.

FIG. 5B is an illustration of a glioblastoma tumoroid with in situ observation of green fluorescent protein (GFP) resulting from nanoparticle transduction via perfusion.

FIG. 6 is a graph illustrating concentration of IL-8 (pg/mL; w-axis) produced by osteosarcoma tumoroids. In this study, the tumoroid is cultured in the presence of ELISA beads which detect IL-8, which are used to measure the local concentration of osteosarcoma tumor-secreted IL-8. The radial distance of the ELISA beads from the tumoroid is noted on the x-axis. Spatiotemporal models were utilized measure tumor production rates of cytokines in real-time. The curve nearest the x-axis corresponds to measurements at 100 minutes, the curve above corresponds to measurements at 200 minutes, and the top curve corresponds to measurements taken at 500 minutes.

FIGS. 7A-7C are bar graphs illustrating cytokine production (pg/mL) in GL261, SMA, Kluc, and CT2A tumor cells (cultured in 2D) which were not exposed to nanoparticles (left bar), exposed to lipid particles lacking RNA (middle bar), and transduced with RNA nanoparticles of the disclosure (right bar). FIG. 7A illustrates IFN-beta production; FIG. 7B illustrates IL-6 production; and FIG. 7C illustrates CCL4 production.

FIGS. 8A-8C are bar graphs illustrating cytokine production (pg/mL) in GL261, SMA, Kluc, and CT2A tumor cells (cultured in 3D) which were not exposed to nanoparticles (left bar), exposed to lipid particles lacking RNA (middle bar), transduced with RNA nanoparticles of the disclosure (right bar). FIG. 8A illustrates IFN-beta production; FIG. 8B illustrates IL-6 production; and FIG. 8C illustrates CCL4 production.

### DETAILED DESCRIPTION

The disclosure provides a method of identifying a tumor for immunotherapy or characterizing a tumor for responsiveness to an immunotherapy. The method, in various aspects, comprises culturing tumor cells obtained from the subject; exposing the tumor cells to nanoparticles comprising a positively-charged surface and an interior comprising (i) a core and (ii) at least two nucleic acid layers, wherein each nucleic acid layer is positioned between a cationic lipid bilayer; and measuring interferon-alpha, CCL4, and/or IL-6 produced by the tumor cells. In various aspects, the method comprises measuring interferon-alpha and IL-6 produced by the tumor cells, and optionally further comprises measuring CCL4 (e.g., the method comprises measuring all three of interferon-alpha, CCL4, and IL-6 produced by the tumor cells). In alternative aspects, the method comprises culturing tumor cells obtained from a subject; exposing the tumor cells to nanoparticles comprising a positively-charged surface and an interior comprising (i) a core and (ii) at least two nucleic acid layers, wherein each nucleic acid layer is positioned between a cationic lipid bilayer; and measuring interferon-alpha, C-X-C Motif Chemokine Ligand 10 (CXCL10), interferon-alpha, and/or interleukin 6 (IL-6) produced by the tumor cells. The method may further include measuring CCL4.

Type I interferons, such as interferon-alpha and interferon-beta, are cytokines associated with antiviral effects produced in response pattern recognition receptors activation (McNab et al., Nat Rev Immunol, 15, 87-103 (2015)). In cancer, type 1 interferons have been attributed for their antitumor effects eliciting proliferation and activity of host immune cells (Ferrantini et al., Biochimie, 89(6-7), 884-93 (2007 June-July)). The instant disclosure contemplates measuring a type 1 interferon (e.g., interferon-alpha and/or interferon beta). Interleukin-6 is a multifunctional cytokine responsible for regulating acute phase protein synthesis, growth and regulation of B lymphocytes and production of neutrophils in bone marrow (Velazquez-Salinas et al., Frontiers in Microbiology, 10, 1057 (2019); Castell et al., FEBS Lett., 242(2), 237-9 (1989 January)). CXCL10 (also known as IP-10) is an inflammatory chemokine induced by IFN-γ that chemoattracts immune cells including natural killer and T lymphocytes (Liu et al., Oncology letters, 2(4), 583-589 (2011)), and restricts blood vessel growth. Chemokine (C-C motif) ligands 4 (CCL4), previously known as macrophage inflammatory protein (MIP-1beta), is a cytokine which acts as a chemoattractant for immune cells (including natural killer cells and monocytes) in inflamed or damaged tissue (Menten et al., Cytokine & Growth Factor Reviews, 13(6), 455-481 (2022)). An increase in the production of the cytokines referenced herein (interferon-alpha, CXCL10, CCL4, interferon-beta, and/or IL-6 (e.g., interferon-alpha, CCL4, and IL-6)) in response to exposure to the nucleic acid-containing nanoparticle compared to the level of cytokine produced by the tumor cells in the absence of the nucleic acid-containing nanoparticle indicates that the tumor cell is responsive to immunotherapy. The method of the disclosure provides an efficient option for screening subjects, or tumors within a subject, to identify patients (or tumors) which will respond to immunotherapy treatment.

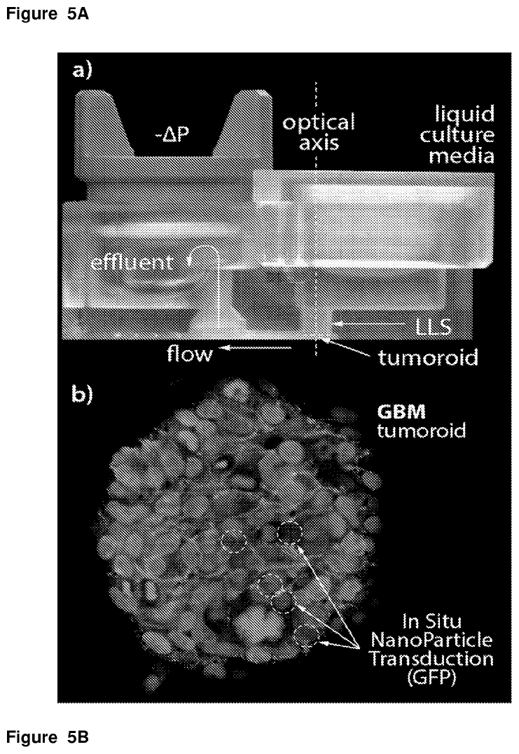

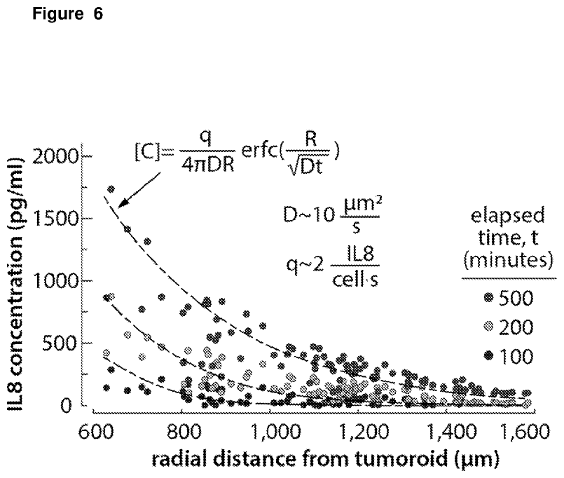

Any method of obtaining tumor cells from a subject may be utilized in the context of the disclosure. A sample comprising blood or other liquids of biological origin or solid tissue such as a biopsy specimen may be obtained by any suitable method (e.g., surgical resection, laparoscopic or needle biopsy, blood or lymph draws, etc.). Samples may be processed and, if needed, enriched for tumor cell populations. The tumor cells are cultured under conditions which support the growth and propagation of the cells for a period of time suitable to characterize chemokine production in response to the nanoparticles. Cell culture techniques are well understood in the art. General culture conditions for mammalian cells are disclosed in, e.g. Animal cell culture: A Practical Approach, D. Rickwood, ed., Oxford University Press, New York (1992). In some examples, the media is used which comprises organoid growth media. In various aspects, the tumor cells are cultured under conditions which allow organoid formation. The term “organoid” refers to a three-dimensional growth of tumor tissue in culture that retains characteristics of the tumor in vivo, e.g., recapitulation of cellular and tissue ultrastructure, immune cell interactions, etc. In this regard, the tumor cells may be maintained in gel substrate, such as a collagen gel solution or a matrigel solution. Cells may be cultured in three dimensions using, e.g., patient derived tumoroids. Tumoroids may be grown from few cells derived from a subject. The resulting tumoroids are capable of being transfected with the nanoparticles described herein and producing cytokines in response to exposure to the nanoparticles. Cytokine production is then measured. See FIGS. 5A-B (illustrating transfection) and FIG. 6 (illustrating cytokine measurement).

The method of the disclosure comprises exposing the tumor cells to nanoparticles comprising a positively-charged surface and an interior comprising (i) a core and (ii) at least two nucleic acid layers, wherein each nucleic acid layer is positioned between a cationic lipid bilayer. Nanoparticles are described further herein. The method further comprises measuring interferon-alpha, C-X-C Motif Chemokine Ligand 10 (CXCL10), Chemokine (C-C motif) ligands 4 (CCL4), interferon-beta, and/or interleukin 6 (IL-6) produced by the tumor cells following exposure to the nanoparticles. Interleukin 8 (IL-8) may be measured. The tumor cells may be exposed to the nanoparticles for any suitable length of time to elicit a response to the nanoparticles. For example, the tumor cells may be exposed to the nanoparticles for three hours, six hours, 12 hours, 18 hours, 24 hours, 30 hours, 36 hours, 42 hours, or 48 hours (or longer). In various aspects, the tumor cells are exposed to the nanoparticles for about six hours to about 48 hours, or for about 12 to about 36 hours, or for about 18 hours to about 30 hours, or for about 20 hours to about 24 hours. After a suitable time of exposure, the amount of cytokine produced by the tumor cells is measured using any suitable method, e.g., RT-PCR, qRT-PCR, QT-PCR oligonucleotide array, Western blot, and/or enzyme-linked immunosorbent assay (ELISA). In various aspects, the method comprises measuring interferon-alpha, optionally in combination with CXCL10 or interferon-beta or IL-6 (or combinations thereof). In various aspects, the method comprises measuring interferon-alpha, CCL4, and/or IL-6 (e.g., the method comprises measuring all of interferon-alpha, CCL4, and IL-6). In various aspects, the method comprises measuring CXCL10, optionally in combination with interferon-alpha or interferon-beta or IL-6 (or combinations thereof). For example, the disclosure contemplates measuring interferon-alpha and CXCL10 or interferon-alpha and interferon-beta. In various aspects, the method comprises measuring interferon-beta, optionally in combination with interferon-alpha or CXCL10 or IL-6 (or combinations thereof). In various aspects, the method comprises measuring IL-6, optionally in combination with interferon-alpha or CXCL10 or interferon-beta (or combinations thereof). For example, the disclosure contemplates measuring IL-6 and interferon-alpha, optionally in combination with CCL4. In various aspects, the method comprises measuring all of interferon-alpha, CXCL10, interferon-beta, and IL-6.

An increase in the production of interferon-alpha, CXCL10, interferon-beta, CCL4, and/or IL-6 after exposure to the nucleic acid-containing nanoparticle compared to the level of cytokine produced by the tumor cells in the absence of the nucleic acid-containing nanoparticle indicates that the tumor cell is responsive to immunotherapy. The level of increase is optionally at least about 25%, at least about 50%, at least about 75%, or at least about 100% (or more) compared to the level of cytokine produced by the tumor cells without exposure to the nanoparticles of the disclosure (i.e., before the nanoparticles are exposed to the tumor cells, or in a control sample cultured concurrently but lacking exposure to the nanoparticles). The ability to differentiate an immunologically “cold” tumor (e.g., a tumor which is not recognized by the immune system) from an immunologically “hot” tumor (e.g., a tumor recognized by the immune system) represents an advancement in the art. Immunological treatment of “cold” tumors presents a great challenge due, at least in part, to the absence of an adaptive immune response. The methods of the disclosure provide a means to identify subjects or tumors responsive to immune checkpoint inhibitors (ICIs) and immunotherapy generally, allowing a clinician to tailor treatment to subjects which will respond.

### Nanoparticles

The method of the disclosure comprises exposing the tumor cells to nanoparticles comprising a positively-charged surface and an interior comprising (i) a core and (ii) at least two nucleic acid layers, wherein each nucleic acid layer is positioned between a cationic lipid bilayer. As used herein the term “nanoparticle” refers to a particle that is less than about 1000 nm in diameter. As the nanoparticles of the present disclosure comprise cationic lipids that have been processed to induce liposome formation, the presently disclosed nanoparticles in various aspects comprise liposomes. Liposomes are artificially-prepared vesicles which, in exemplary aspects, are primarily composed of a lipid bilayer. In various aspects the liposomes of the present disclosure are of different sizes and the composition may comprise one or more of (a) a multilamellar vesicle (MLV) which may be hundreds of nanometers in diameter and may contain a series of concentric bilayers separated by narrow aqueous compartments, (b) a small unicellular vesicle (SUV) which may be smaller than, e.g., 50 nm in diameter, and (c) a large unilamellar vesicle (LUV) which may be between, e.g., 50 and 500 nm in diameter.

The nanoparticle comprises a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle comprises at least three nucleic acid layers, each of which is positioned between a cationic lipid bilayer. In exemplary aspects, the nanoparticle comprises at least four or five nucleic acid layers, each of which is positioned between a cationic lipid bilayer. In exemplary aspects, the nanoparticle comprises at least more than five (e.g., 6, 7, 8, 9, 10, 11, 12, 13, 14, 15, 16, 17, 18, 19, 20, or more) nucleic acid layers, each of which is positioned between a cationic lipid bilayer. As used herein the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. Optionally, the cationic lipid bilayer comprises, consists essentially of, or consists of DOTAP. In various instances, the cationic lipid bilayer comprises, consists essentially of, or consists of DOTMA. The surface optionally comprises a plurality of hydrophilic moieties of the cationic lipid of the cationic lipid bilayer. In some aspects, the core comprises a cationic lipid bilayer. In various instances, the core lacks nucleic acids, optionally, the core comprises less than about 0.5 wt % nucleic acid.

In exemplary aspects, the nanoparticle has a diameter within the nanometer range and accordingly in certain instances are referred to herein as “nanoliposomes” or “liposomes.” In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, or about 400 nm to about 500 nm. In exemplary aspects, the nanoparticle has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm 5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, or about 300 nm±5 nm. In exemplary aspects, the nanoparticle is about 50 nm to about 250 nm in diameter. In some aspects, the nanoparticle is about 70 nm to about 200 nm in diameter.

In exemplary aspects, the nanoparticle is present in a composition comprising a heterogeneous population of nanoparticles ranging in diameter, e.g., about 50 nm to about 500 nm or about 50 nm to about 250 nm in diameter. Optionally, the composition comprises a heterogeneous population of nanoparticles ranging from about 70 nm to about 200 nm in diameter.

In exemplary instances, the nanoparticle is characterized by a zeta potential of about +40 mV to about +60 mV, e.g., about +40 mV to about +55 mV, about +40 mV to about +50 mV, about +40 mV to about +50 mV, about +40 mV to about +45 mV, about +45 mV to about +60 mV, about +50 mV to about +60 mV, about +55 mV to about +60 mV. In exemplary aspects, the nanoparticle has a zeta potential of about +45 mV to about +55 mV. The nanoparticle, in various instances, has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1): e1256527 (2016).

In exemplary embodiments, the nanoparticle comprises a cationic lipid. In some embodiments, the cationic lipid is a low molecular weight cationic lipid such as those described in U.S. Patent Application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

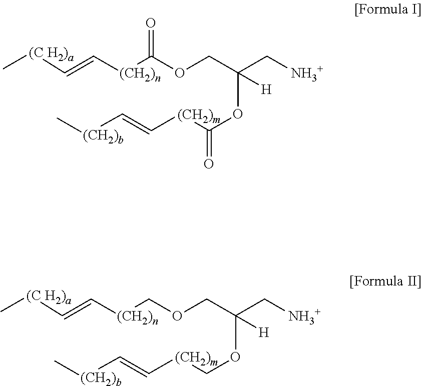

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles comprise liposomes formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (U.S. Patent Publication No. 20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo. The nanoparticles in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al., Pharm Res. 2005; 22(3):362-72. In exemplary instances, the liposome comprises 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al., J. Control Release 2005; 107(2): 276-87.

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0% and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example, a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Patent Publication No. 20130150625, herein incorporated by reference in its entirety. As a non-limiting example, the cationic lipid in certain aspects is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the liposome comprises from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In various aspects, the nanoparticle does not comprise a neutral lipid. In some embodiments, the formulation includes from about 5% to about 50% on a molar basis of the sterol (e.g., about 15 to about 45%, about 20 to about 40%, about 40%, about 38.5%, about 35%, or about 31% on a molar basis). An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In exemplary aspects, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N,N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N, N-dimethylnonacosa-11,20,2-trien-10-amine or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Patent Publication No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In various aspects, the cationic liposomes optionally do not comprise a non-cationic lipid. Neutral molecules, in some aspects, may interfere with coiling/condensation of multi-lamellar nanoparticles resulting in RNA loaded liposomes greater than 200 nm in size. Cationic liposomes generated without helper molecules can comprise a size of about 70-200 nm (or less). These constructs consist essentially of a cationic lipid with negatively charged nucleic acid, and may be formulated in a sealed rotary vacuum evaporator which prevents oxidation of the particles (when exposed to the ambient environment). In this embodiment, the absence of a helper lipid optimizes mRNA coiling into tightly packaged multilamellar NPs where each NP contains a greater amount of nucleic acid per particle. Due to increased nucleic acid payload per particle, these multi-lamellar RNA nanoparticles drive significantly greater innate immune responses, which are a significant predictor of efficacy for modulating the immune system.

In some aspects, the nucleic acid molecules are present at a nucleic acid molecule:cationic lipid ratio of about 1 to about 5 to about 1 to about 25. In some aspects, the nucleic acid molecules are present at a nucleic acid molecule:cationic lipid ratio of about 1 to about 5 to about 1 to about 20, optionally, about 1 to about 15, about 1 to about 10, or about 1 to about 7.5. As used herein, the term “nucleic acid molecule:cationic lipid ratio” is meant a mass ratio, where the mass of the nucleic acid molecule is relative to the mass of the cationic lipid. Also, in exemplary aspects, the term “nucleic acid molecule:cationic lipid ratio” is meant the ratio of the mass of the nucleic acid molecule, e.g., RNA, added to the liposomes comprising cationic lipids during the process of manufacturing the ML RNA NPs of the present disclosure. In exemplary aspects, the nanoparticle comprises less than or about 10 pg RNA molecules per 150 pg lipid mixture. In exemplary aspects, the nanoparticle is made by incubating about 10 pg RNA with about 150 pg liposomes. In alternative aspects, the nanoparticle comprises more RNA molecules per mass of lipid mixture. For example, the nanoparticle may comprise more than 10 pg RNA molecules per 150 pg liposomes. The nanoparticle in some instances comprises more than 15 pg RNA molecules per 150 pg liposomes or lipid mixture.

In various aspects, the nucleic acid molecules are RNA molecules, e.g., transfer RNA (tRNA), ribosomal RNA (rRNA), or messenger RNA (mRNA). In various aspects, the RNA molecules comprise tRNA, rRNA, mRNA, or a combination thereof. In various aspects, the RNA in the nanoparticle exposed to the tumor cell to determine responsiveness to immunotherapy is mRNA, which may or may not encode a protein. In various aspects, the RNA is total RNA isolated from a cell. In exemplary aspects, the RNA is total RNA isolated from a diseased cell, such as, for example, a tumor cell or a cancer cell. Methods of obtaining total tumor RNA is known in the art and described herein at Example 1.

In various aspects, the method comprises administering a population of second nanoparticles to the subject. The features described above with respect to nanoparticles also applies to the second nanoparticles. The second nanoparticles may be compositionally the same as the nanoparticles exposed to the tumor cells, or the second nanoparticles may be compositionally different (e.g., comprise different nucleic acid(s)). Additional features of nanoparticles, which may apply to the nanoparticles exposed to the tumor cells or the second nanoparticles administered to the subject, are provided below.

In exemplary instances, the RNA molecules of the nanoparticles are mRNA. In various aspects, mRNA is in vitro transcribed mRNA. In various instances, the mRNA molecules are produced by in vitro transcription (IVT). Suitable techniques of carrying out IVT are known in the art. In exemplary aspects, an IVT kit is employed. In exemplary aspects, the kit comprises one or more IVT reaction reagents. As used herein, the term “in vitro transcription (IVT) reaction reagent” refers to any molecule, compound, factor, or salt, which functions in an IVT reaction. For example, the kit may comprise prokaryotic phage RNA polymerase and promoter (T7, T3, or SP6) with eukaryotic or prokaryotic extracts to synthesize proteins from exogenous DNA templates. Optionally, the RNA is in vitro transcribed mRNA, wherein the in vitro transcription template is cDNA made from RNA extracted from a tumor cell. In various aspects, the nanoparticle comprises a mixture of RNA which is RNA isolated from a tumor of a human, optionally, a malignant brain tumor, optionally, a glioblastoma, medulloblastoma, diffuse intrinsic pontine glioma, or a peripheral tumor with metastatic infiltration into the central nervous system. In various aspects, the RNA comprises a sequence encoding a poly(A) tail so that the in vitro transcribed RNA molecule comprises a poly(A) tail at the 3′ end. In various aspects, the method of making a nanoparticle comprises additional processing steps, such as, for example, capping the in vitro transcribed RNA molecules.

The RNA (e.g., mRNAs) in exemplary aspects encode a protein. Optionally, the protein is selected from the group consisting of a tumor antigen, a cytokine, and a co-stimulatory molecule. Indeed, the protein is, in some aspects, selected from the group consisting of a tumor antigen, a co-stimulatory molecule, a cytokine, a growth factor, a hematopoietic factor, or a lymphokine, including, e.g., cytokines and growth factors that are effective in inhibiting tumor metastasis, and cytokines or growth factors that have been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof. In exemplary aspects, the tumor antigen is an antigen derived from a viral protein, an antigen derived from point mutations, or an antigen encoded by a cancer-germline gene. In exemplary aspects, the tumor antigen is pp65, p53, KRAS, NRAS, MAGEA, MAGEB, MAGEC, BAGE, GAGE, LAGE/NY-ESO1, SSX, tyrosinase, gp100/pmel17, Melan-A/MART-1, gp75/TRP1, TRP2, CEA, RAGE-1, HER2/NEU, WT1. In exemplary aspects, the co-stimulatory molecule is selected from the group consisting of CD80 and CD86. In some aspects, the protein is not expressed by a tumor cell or by a human. In exemplary instances, the protein is not related to a tumor antigen or cancer antigen. In some aspects, the protein is non-specific relative to a tumor or cancer. For example, the non-specific protein may be green fluorescence protein (GFP) or ovalbumin (OVA). In various aspects, the nucleic acid does not encode a protein.

In various aspects, the nucleic acid layers comprise a sequence of a nucleic acid molecule expressed by slow-cycling cells (SCCs). The term “slow-cycling cells” or “SCCs” refers to tumor or cancer cells that proliferate at a slow rate. In exemplary aspects, the SCCs have a doubling time of at least about 50 hours. SCCs have been identified in numerous cancer tissues, including, melanoma, ovarian cancer, pancreatic adenocarcinoma, breast cancer, glioblastoma, and colon cancer. As taught in Deleyrolle et al., Brain 134(5): 1331-1343 (2011) (incorporated by reference herein, particularly with respect to the description of SCCs), SCCs display increased tumor-initiation properties and are stem cell like. Because of their slow proliferation rate, SCCs are also referred to as label-retaining cells (LRCs). In exemplary instances, the nucleic acid molecules are RNA extracted from isolated SCCs or are nucleic acid molecules which hybridize to RNA extracted from isolated SCCs. Optionally, the SCCs are isolated from a mixed tumor cell population obtained from a subject with a tumor (e.g., a glioblastoma). As used herein, the term “mixed tumor cell population” refers to a heterogeneous cell population comprising tumor cells of different sub-types and comprising slow-cycling cells and at least one other tumor cell type, e.g., fast-cycling cells (FCCs).

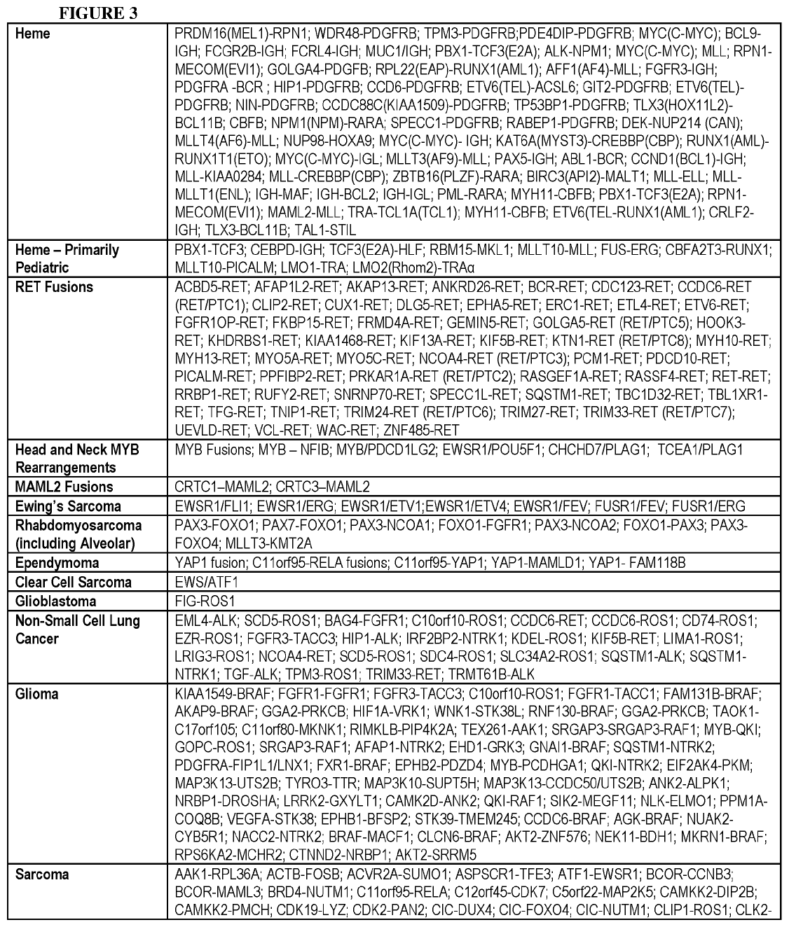

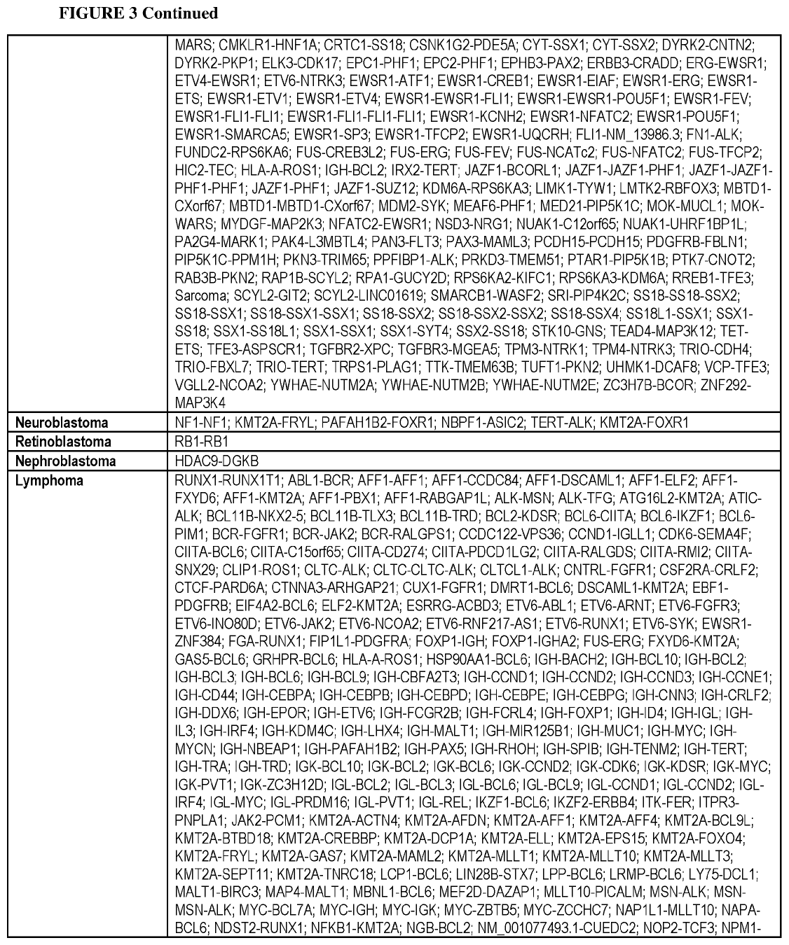

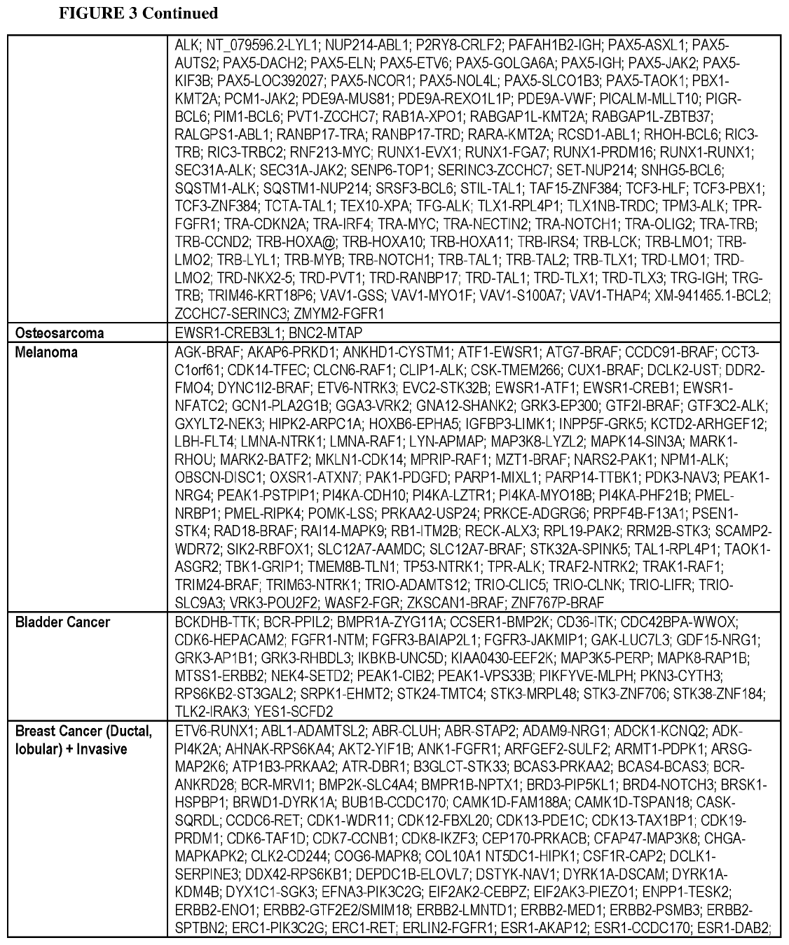

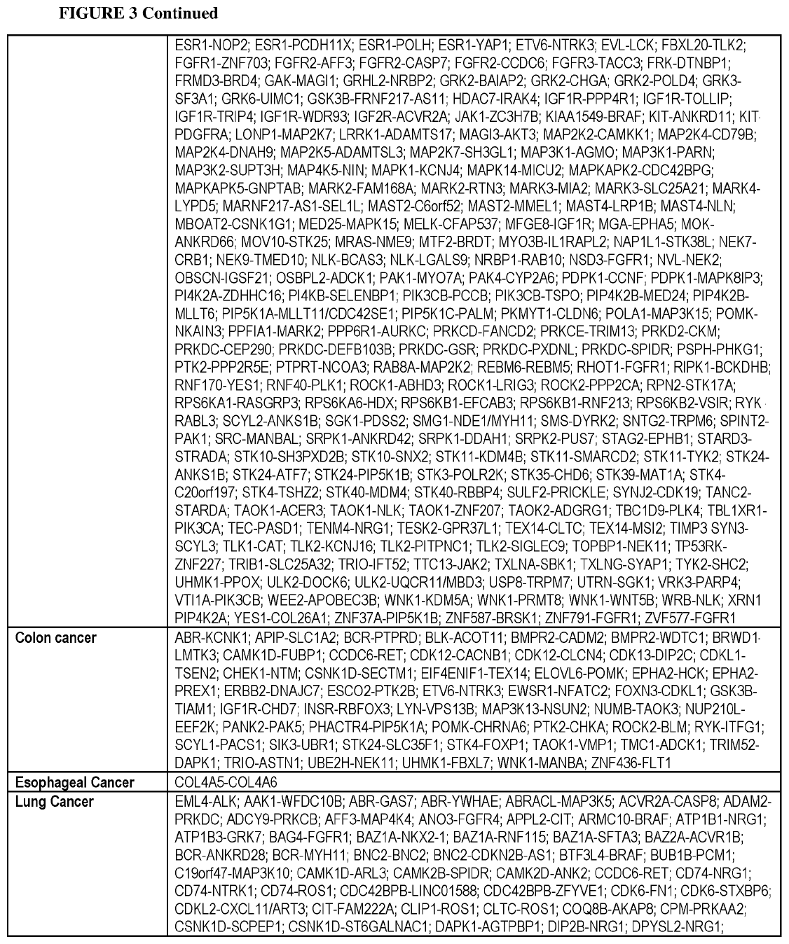

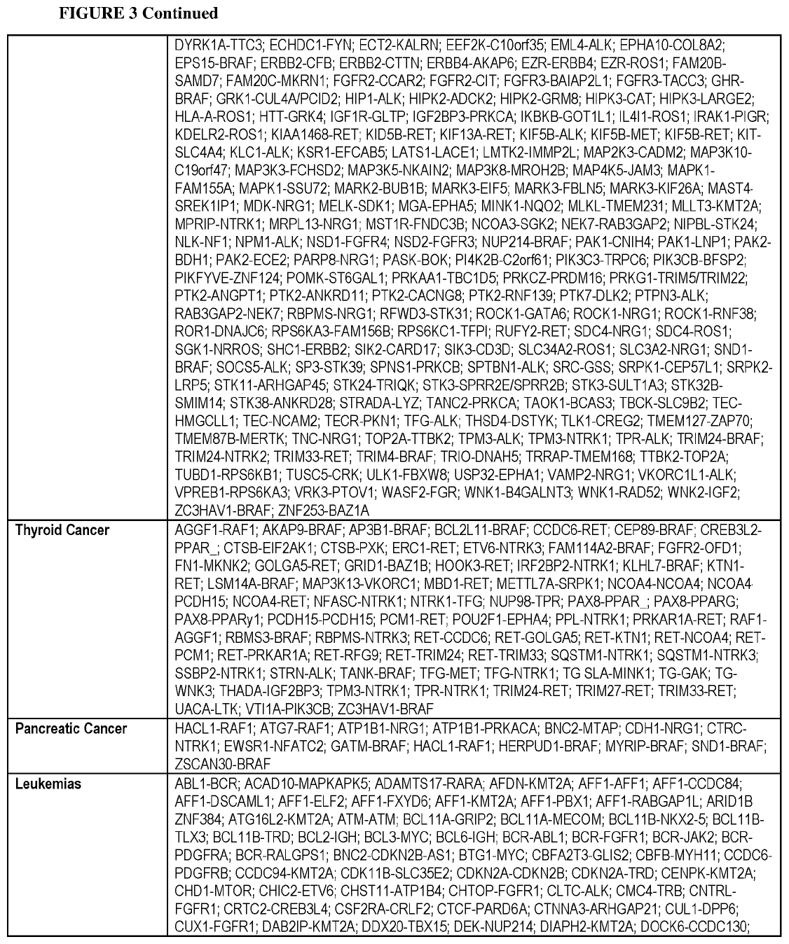

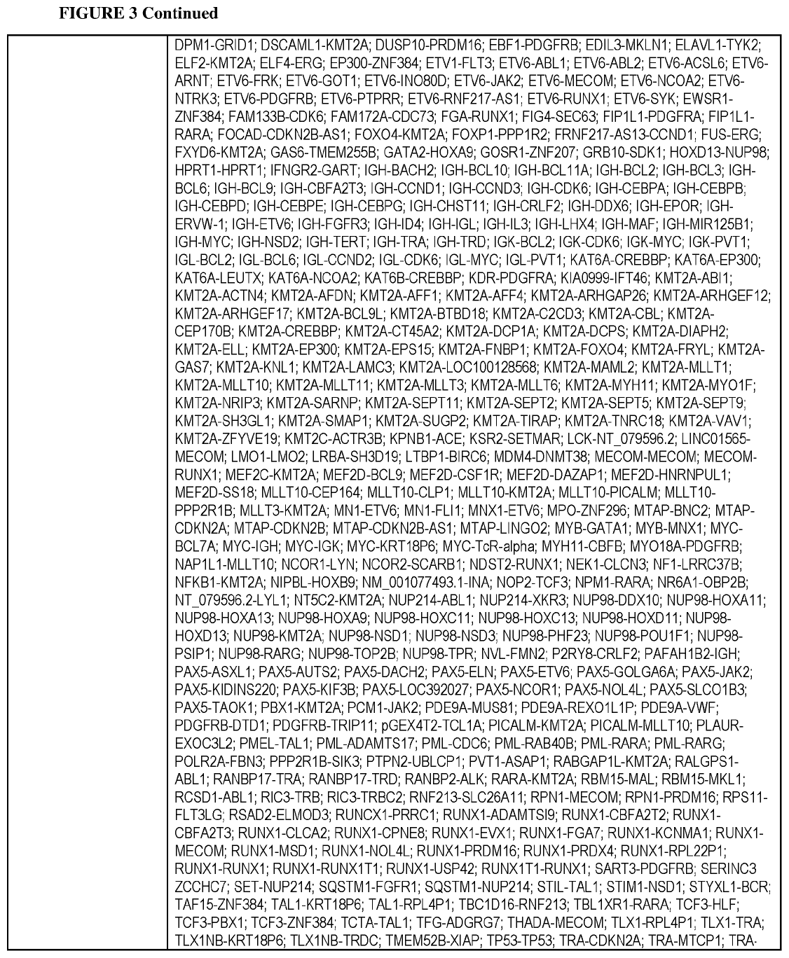

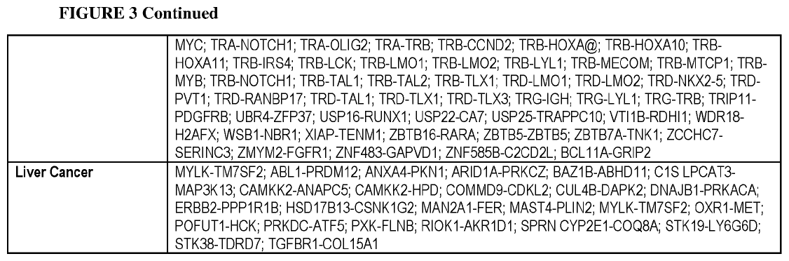

In exemplary instances, the nanoparticle comprises a mixture or plurality of different RNA molecules expressed by SCCs. In certain instances, the mixture or plurality comprises at least 10 (e.g., at least 20, at least 30, at least 40, at least 50, at least 60, at least 70, at least 80, at least 90) different RNA molecules expressed by SCCs. In some aspects, the mixture or plurality comprises at least 100 (e.g., at least 150, at least 200, at least 250, at least 300, at least 350, at least 400, at least 450, at least 500, at least 550, at least 600, or more (e.g., at least 700, at least 800 at least 900)) different RNA molecules expressed by SCCs. In aspects, the nanoparticles comprise a mixture or plurality of RNA molecules which represent at least in part the transcriptome of SCCs. The term “transcriptome” refers to the sum total of all the messenger RNA molecules expressed from the genes of an organism. The term “SCC transcriptome” refers to the sum total of all the mRNA molecules expressed by SCCs. In particular instances, the SCC transcriptome is produced by first isolating total RNA from the tumor cells, which total RNA is then used to generate cDNA by RT-PCR using routine methods. The cDNA may be used to synthesize protected mRNA transcripts (e.g., 7-methyl guanosine capped RNA) using, for example, an Ambion® mMESSAGE mMACHINE® transcription kit. In exemplary aspects, the SCC transcriptome is the sum total of all the mRNA expressed from the genes listed in FIG. 3. In alternative or additional embodiments, the nucleic acid molecules of the nanoparticles, e.g., the RNA, are de novo synthesized RNA encoded by at least two (e.g., at least 3, at least 4, at least 5, at least 6, at least 7, at least 8, at least 9) different genes listed in FIG. 3. In exemplary instances, the nucleic acid molecules are RNA encoded by at least 10 (e.g., at least 20, at least 30, at least 40, at least 50, at least 60, at least 70, at least 80, at least 90) different genes listed in FIG. 3. In some aspects, the nucleic acid molecules are RNA encoded by at least 100 (e.g., at least 150, at least 200, at least 250, at least 300, at least 350, at least 400, at least 450, at least 500, at least 550, at least 600, or more (e.g., at least 700, at least 800 at least 900)) different genes listed FIG. 3.

In various instances, the RNA molecules are antisense molecules, optionally siRNA, shRNA, miRNA, or any combination thereof. The antisense molecule can be one which mediates RNA interference (RNAi). As known by one of ordinary skill in the art, RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et al., Cell. 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001)). It has been reported that introduction of dsRNA into mammalian cells does not result in efficient Dicer-mediated generation of siRNA and therefore does not induce RNAi (Caplen et al., Gene 252, 95-105 (2000); Ui-Tei et al., FEBS Lett, 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411, 494-498 (2001)).

In this regard, the RNA molecule in some aspects mediates RNAi and in some aspects is a siRNA molecule specific for inhibiting the expression of a protein. The term “siRNA” as used herein refers to an RNA (or RNA analog) comprising from about 10 to about 50 nucleotides (or nucleotide analogs) which is capable of directing or mediating RNAi. In exemplary embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the RNA molecule is alternatively a short hairpin RNA (shRNA) molecule specific for inhibiting the expression of a protein. The term “shRNA” as used herein refers to a molecule of about 20 or more base pairs in which a single-stranded RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template.

In exemplary aspects, the antisense molecule is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, NY, 2011; Bader and Lammers, “The Therapeutic Potential of microRNAs” Innovations in Pharmaceutical Technology, pages 52-55 (March 2011).

In certain instances, the RNA molecule is an antisense molecule, optionally, an siRNA, shRNA, or miRNA, which targets a protein of an immune checkpoint pathway for reduced expression. In various aspects, the protein of the immune checkpoint pathway is CTLA-4, PD-1, PD-L1, PD-L2, B7-H3, B7-H4, TIGIT, LAG3, CD112 TIM3, BTLA, or co-stimulatory receptor ICOS, OX40, 41BB, or GITR. The protein of the immune-checkpoint pathway in certain instances is CTLA4, PD-1, PD-L1, B7-H3, B7H4, or TIM3. Immune checkpoint signaling pathways are reviewed in Pardoll, Nature Rev Cancer 12(4): 252-264 (2012), incorporated herein by reference in its entirety.

In exemplary embodiments, the nanoparticles of the present disclosure comprise a mixture of RNA molecules. In exemplary aspects, the mixture of RNA molecules is RNA isolated from cells from a human and optionally, the human has a tumor. In some aspects, the mixture of RNA is RNA isolated from the tumor of the human. In exemplary aspects, the human has cancer, optionally, any cancer described herein. Optionally, the tumor from which RNA is isolated is selected from the group consisting of a glioma (including, but not limited to, a glioblastoma), a medulloblastoma, a diffuse intrinsic pontine glioma, or a peripheral tumor with metastatic infiltration into the central nervous system (e.g., melanoma or breast cancer). In exemplary aspects, the tumor from which RNA is isolated is a tumor of a cancer, e.g., any of the cancers described herein.

In various aspects, the nanoparticles comprise a nucleic acid molecule (e.g., RNA molecule) comprising a nucleotide sequence encoding a chimeric protein comprising a LAMP protein. In certain aspects, the LAMP protein is a LAMP1, LAMP 2, LAMP3, LAMP4, or LAMP5 protein.

The nanoparticles of the disclosure may be produced by any suitable method, such as a method comprising the following steps: (A) mixing nucleic acid molecules and liposomes at a nucleic acid (e.g., RNA):liposome ratio of about 1 to about 5 to about 1 to about 25, such as about 1 to 5 to about 1 to about 20, optionally, about 1 to about 15, to obtain nucleic acid—(e.g., RNA) coated liposomes; and (B) mixing the RNA-coated liposomes with a surplus amount of liposomes. The liposomes are made by a process of making liposomes comprising drying a lipid mixture comprising a cationic lipid and an organic solvent by evaporating the organic solvent under a vacuum. A description of an exemplary method of making a nanoparticle comprising a positively-charged surface and an interior comprising (i) a core and (ii) at least two nucleic acid layers, wherein each nucleic acid layer is positioned between a cationic lipid bilayer is provided herein at Example 1. Any one or more of the steps described in Example 1 may be included. For instance, the method may comprise one or more steps required for preparing the RNA prior to being complexed with the liposomes.

In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

Provided herein are compositions comprising a nanoparticle of the present disclosure and a pharmaceutically acceptable carrier, excipient or diluent. In exemplary aspects, the composition is a pharmaceutical composition comprising a plurality of nanoparticles according to the present disclosure and a pharmaceutically acceptable carrier, diluent, or excipient and intended for administration to a human. In exemplary aspects, the composition is a sterile composition. In exemplary instances, the composition comprises a plurality of nanoparticles of the present disclosure. Optionally, at least 50% of the nanoparticles of the plurality have a diameter between about 100 nm to about 250 nm, although higher diameters (e.g., 500 nm) also are contempated. In various aspects, the composition comprises about 10¹⁰ nanoparticles per mL to about 10¹⁵ nanoparticles per mL, optionally about 10¹² nanoparticles±10% per mL.

In exemplary aspects, the composition may comprise additional components other than the nanoparticle. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous routes. Suitable routes include intravenous, intradermal, intramuscular, intraperitoneal, intranodal and intrasplenic, for example.

### Subjects

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less. In exemplary aspects, the subject has a DMG. In various instances, the DMG is diffuse intrinsic pontine glioma (DIPG).

A subject may be one who has been previously diagnosed with or identified as suffering from or having a condition in need of treatment (e.g., cancer) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition. The subject, in various aspects, has previously received a treatment or therapy for the condition (e.g., previously been administered an anti-cancer therapy). In various aspects, the subject is an immunotherapy naïve patient, i.e., a patient who has not previously been treated with the immunotherapy prior to method disclosed herein. In other aspects, the disclosed method is performed during the course of treatment of the subject, such as after the administration of one or more doses of immunotherapeutic, optionally wherein the subject is not sufficiently responding to the immunotherapy treatment. In this scenario, the disclosed method may be employed, e.g., to elucidate the reasons why a subject is not responding as expected to a particular immunotherapy.

### Therapy

Without being bound to any particular theory, the data provided herein support the use of the presently disclosed nanoparticles for identifying subjects (or tumors or malignancies within a subject) which will respond to an immunotherapy. The immunotherapy may include, but is not limited to, immune checkpoint inhibitors, described further below. In various aspects, the method of the disclosure further comprises administering the immunotherapy to the subject. Alternatively or in addition, the method comprises administering to the subject a population of second nanoparticles described herein to elicit an immune response to the tumor.

An “immune checkpoint inhibitor” or “IC” is any agent (e.g., compound or molecule) that that decreases, blocks, inhibits, abrogates or interferes with the function of a protein of an immune checkpoint pathway. Proteins of the immune checkpoint pathway regulate immune responses and, in some instances, prevent T cells from attacking cancer cells. In various aspects, the protein of the immune checkpoint pathway is, for example, CTLA-4, PD-1, PD-L1, PD-L2, B7-H3, B7-H4, TIGIT, VISTA, LAG3, CD112 TIM3, BTLA, or co-stimulatory receptor ICOS, OX40, 41 BB, or GITR. In various aspects, the ICI is a small molecule, an inhibitory nucleic acid, or an inhibitor polypeptide. In various aspects, the ICI is an antibody, antigen-binding antibody fragment, or an antibody protein product, that binds to and inhibits the function of the protein of the immune checkpoint pathway. Suitable ICIs which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune) (Wei et al., Cancer Discovery 8: 1069-1086 (2018)). Other examples of ICIs include, but are not limited to, IMP321 (LAG3; Eftilagimod alpha; Immuntep); BMS-986016 (LAG3; relatlimab; Bristol Meyers Squibb); IPH2101 (KIR; Innate Pharma); tremelimumab (CTLA-4; Medimmune); pidilizumab (PD-1; Medivation); AUNP12 (PD-1; a branched 29-amino acid peptide sequence engineered from the PD-L1/L2 binding domain of PD-1; Aurigene); MGA271 (B7-H3; enoblituzumab; MacroGenics); and TSR-022 (TIM3; cobolimab; Tesaro).

In various aspects, the ICI is a PD-L1 inhibitor. Programmed death-ligand 1 (PD-L1; also known as cluster of differentiation 274 (CD274) or B7 homolog 1 (B7-H1)) is a transmembrane protein that functions to suppress the immune system in, e.g., pregnancy, tissue allografts, and autoimmune disease. Binding of PD-L1 to its receptor PD-1 transmits an inhibitory signal that reduces the proliferation and function of T cells and can induce apoptosis. For example, the PD-L1 inhibitor binds to and inhibits the function of PD-L1. In various aspects, the PD-L1 inhibitor is an anti-PD-L1 antibody, antigen binding antibody fragment, or an antibody-like molecule.

In various aspects, the ICI is a PD-1 inhibitor. “Programmed Death-1” (PD-1), also known as cluster of differentiation 279 (CD279), refers to an immunoinhibitory receptor belonging to the CD28 family. PD-1 is expressed on previously activated T cells in vivo, and binds to two ligands, PD-L1 and PD-L2. The human PD-1 sequence can be found under GenBank Accession No. U64863. For example, the PD-1 inhibitor binds to and inhibits the function of PD-1, e.g., an anti-PD-1 antibody, antigen binding antibody fragment, or an antibody-like molecule. In various aspects, the PD-1 inhibitor is durvalumab, atezolizumab, or avelumab. In various aspects, the ICI is a PD-L2 inhibitor. For example, the PD-L2 inhibitor binds to and inhibits the function of PD-L2, e.g., an anti-PD-L2 antibody, antigen binding antibody fragment, or an antibody-like molecule.

Examples of PD-1 and PD-L1 inhibitors are described in, e.g., U.S. Pat. Nos. 7,488,802; 7,943,743; 8,008,449; 8,168,757; 8,217,149; and PCT Patent Publication Nos. WO03042402, WO2008156712, WO2010089411, WO2010036959, WO2011066342, WO2011159877, WO2011082400, and WO2011161699; which are incorporated by reference herein in their entireties.

Cytotoxic T-lymphocyte-associated protein 4 (CTLA-4, also known as CD152), is a membrane protein expressed on T cells and regulatory T cells (Treg). CTLA-4 binds B7-1 (CD80) and B7-2 (CD86) on antigen-presenting cells (APC), which inhibits the adaptive immune response. In humans, CTLA-4 is encoded in various isoforms; an exemplary amino acid sequence is available as GenBank Accession No. NP_001032720. A representative anti-CTLA-4 antibody is ipilimumab (YERVOY®, Bristol-Myers Squibb).

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody may be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects, the ICI is an antigen binding antibody fragment, e.g., a Fab, Fc, F(ab′)₂, or a pFc′. The architecture of antibodies has been exploited to create a growing range of alternative antibody formats that spans a molecular-weight range of at least or about 12-150 kDa and a valency (n) range from monomeric (n=1), dimeric (n=2) and trimeric (n=3) to tetrameric (n=4) and potentially higher; such alternative antibody formats are referred to herein as “antibody-like molecules”. Antibody-like molecules can be an antigen binding format based on antibody fragments, e.g., scFvs, Fabs and VHH/VH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab are widely used fragments that can be easily produced in prokaryotic hosts. Other antibody-like molecules include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody-like molecule. The structure of a peptibody consists of a biologically active peptide grafted onto an Fc domain. Peptibodies are well-described in the art. See, e.g., Shimamoto et al., mAbs 4(5): 586-591 (2012). Other antibody-like molecules include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015). In exemplary aspects, the antibody-like molecule comprises any one of these antibody-like molecules (e.g., scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate).

As used herein, the term “inhibit” and words stemming therefrom does not require a 100% or complete inhibition or abrogation. Rather, there are varying degrees of inhibition of which one of ordinary skill in the art recognizes as having a potential benefit or therapeutic effect. The ICIs may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about an 80% inhibition, at least or about a 90% inhibition, at least or about a 95% inhibition, at least or about a 98% inhibition).

As used herein “sensitivity” refers to the way a tumor reacts to a drug/compound, e.g., an ICI inhibitor (e.g., PD-L1 inhibitor). In exemplary aspects, “sensitivity” means “responsive to treatment” and the concepts of “sensitivity” and “responsiveness” are positively associated in that a tumor or cancer cell that is responsive to a drug/compound treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. In exemplary aspects, “sensitivity” or “responsiveness” is opposite to “resistant” and the concept of “resistance” is negatively associated with “sensitivity”. For example, a tumor that is resistant to a drug treatment is neither sensitive nor responsive to that drug, and that drug is not an effective treatment for that tumor or cancer cell. In the context of ICI's, a tumor which is insensitive to ICIs is one which does not respond to ICI therapy in a clinically significant way. “Sensitivity” also is used herein with respect to a host immune response. In this respect, a tumor which evades a host immune response is “resistant” (or refractory). A tumor that is “sensitive” to a host immune response is recognized by the host immune system and subject to attack by immune effector cells. A tumor that is “sensitive” to a host immune response is recognized by the host immune system and subject to attack by immune effector cells.

In exemplary aspects, the method comprises administering an ICI to the subject. In this regard, the present disclosure further provides a method of treating a subject with cancer. The method comprises culturing tumor cells obtained from the subject; exposing the tumor cells to nanoparticles comprising a positively-charged surface and an interior comprising (i) a core and (ii) at least two nucleic acid layers, wherein each nucleic acid layer is positioned between a cationic lipid bilayer; measuring interferon-alpha, CCL4, CXCL10, interferon-beta, and/or IL-6 produced by the tumor cells (e.g., measuring interferon-alpha and CXCL10; measuring IL-6 and interferon-alpha, or measuring IL-6, CCL4, and interferon-alpha); and administering an immunotherapy (such as an immune checkpoint inhibitor) to the subject. In exemplary aspects, the method further comprises administering to the subject a composition comprising the nanoparticle of the disclosure comprising a cationic lipid and nucleic acid molecules, optionally via systemic administration. The nanoparticle may be any of those described herein. For example, the nanoparticle may comprise DOTAP and the nucleic acid molecules may be a mixture of mRNA expressed by the tumor of the subject. In exemplary aspects, the composition comprising the liposome comprises a heterogeneous mixture of liposomes varied in size, though having a diameter within the range of 50 nm to about 250 nm (although larger diameters also are contemplated, e.g., 500 nm). In exemplary aspects, the liposomes have a zeta potential of about 30 mV to about 60 mV, optionally, about 40 mV to about 50 mV. In exemplary aspects, the ICI is a PD-L1 inhibitor, such as a PD-L1 antibody. PD-L1 inhibitors are known in the art and include, but are not limited to, atezolizumab, avelumab, and durvalumab.

As used herein, the term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the presently disclosure may inhibit one or more symptoms of the disease. Also, the treatment provided by the methods of the present disclosure may encompass slowing the progression of the disease. For example, the methods can treat cancer by virtue of enhancing the T cell activity or an immune response against the cancer, thereby reducing tumor or cancer growth, reducing metastasis of tumor cells, increasing cell death of tumor or cancer cells, and the like. The term “treat” also encompasses delaying the onset or reoccurrence/relapse of the disease being treated.

“Treatment” involves any improvement in the subjects well-being (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about an 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease; (8) decrease in levels of cancer markers; (9) an increased patient survival rate; and/or (10) some relief from one or more symptoms associated with the disease or condition (e.g., pain). For example, the efficacy of treatment may be determined by detecting of a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other immunotherapy treatment or chemotherapy. In various aspects, the methods of the disclosure further comprise monitoring treatment in the subject.

Parenteral dosage forms of any agent described herein can be administered to a subject by various routes, including, but not limited to, epidural, intracerebral, intracerebroventricular, epicutaneous, intraarterial, intraarticular, intracardiac, intracavernous injection, intradermal, intralesional, intramuscular, intraocular, intraosseous infusion, intraperitoneal, intrathecal, intrauterine, intravaginal administration, intravenous, intravesical, intravitreal, subcutaneous, transdermal, perivascular administration, or transmucosal. For administration to the brain, a pharmaceutical composition can be introduced into tumor tissue using an intratumoral delivery catheter, ventricular shunt catheter attached to a reservoir (e.g., Omaya reservoir), infusion pump, or introduced into a tumor resection cavity (such as Gliasite, Proxima Therapeutics). Tumor tissue in the brain also can be contacted by administering a pharmaceutical composition via convection using a continuous infusion catheter or through cerebrospinal fluid. In various instances, a composition of the disclosure is administered to the subject intravenously.

For purposes of the disclosure, the amount or dose of the active agent (i.e., the “effective amount”) administered should be sufficient to achieve a desired biological effect, e.g., a therapeutic or prophylactic response, in the subject over a reasonable time frame. For example, one or more doses of the nanoparticles described herein and/or ICI should be sufficient to, e.g., sensitize a tumor to an immune response (and optionally treat a cancer) in a clinically acceptable period of time e.g., 1 to 20 or more weeks, from the time of first administration. In certain embodiments, the time period could be even longer. By way of example and not intending to limit the present disclosure, the dose of the active agents of the present disclosure can be about 0.0001 to about 1 g/kg body weight of the subject being treated/day, from about 0.0001 to about 0.001 g/kg body weight, or about 0.01 mg to about 1 g/kg body weight.

In instances wherein the method comprises administering a nanoparticle of the disclosure and an ICI to a subject, the nanoparticle composition and ICI may be administered together (in the same formulation or separate formulations administered close in time) or may be administered sequentially (i.e., the nanoparticle composition is administered and the ICI is administered separately at different time points (e.g., hours or days apart)). In this regard, the nanoparticle composition of the disclosure is optionally administered prior to the ICI, e.g., at least about six hours, at least about 12 hours, at least about 18 hours, or at least about 24 hours prior to ICI administration. In this regard, the nanoparticles may be administered at least about three days, one week, two weeks, three weeks, four weeks (i.e., one month), two months, or three months prior to administration of ICI. For example, the method may, in various instances, comprise a first period of nanoparticle treatment followed by a second period of ICI treatment. The second period of ICI treatment may also entail treatment with the nanoparticles to enhance the immune response (e.g., the second period may comprise both ICI administration and nanoparticle administration). The first period of nanoparticle administration may entail multiple doses of nanoparticles administered to the subject over time, e.g., two, three, four, five, or more doses administered over a treatment period of one week, two weeks, three weeks, four weeks, five weeks or six weeks, prior to administration of an ICI.

The cancer treatable by the methods disclosed herein may be any cancer, e.g., any malignant growth or tumor caused by abnormal and uncontrolled cell division that may spread to other parts of the body through the lymphatic system or the blood stream.

The cancer in some aspects is one selected from the group consisting of acute lymphocytic cancer, acute myeloid leukemia, alveolar rhabdomyosarcoma, bone cancer, brain cancer (e.g., glioma), breast cancer (e.g., triple negative breast cancer), cancer of the anus, anal canal, or anorectum, cancer of the eye, cancer of the intrahepatic bile duct, cancer of the joints, cancer of the head, neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), Hodgkin lymphoma, endometrial or hepatocellular carcinoma, hypopharynx cancer, kidney cancer, larynx cancer, liver cancer, lung cancer (e.g., non-small cell lung cancer, bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of head and neck, ovarian, cervical, bladder and oesophageal cancers, pancreatic, gastrointestinal cancer, gastric, breast, endometrial and colorectal cancers, hepatocellular carcinoma, glioblastoma, bladder, and lung cancer (e.g., non-small cell lung cancer (NSCLC), bronchioloalveolar carcinoma). In various aspects, the subject has a solid tumor. Optionally, the subject suffers from a malignant brain tumor, such as a glioblastoma, medulloblastoma, diffuse intrinsic pontine glioma, or a peripheral tumor with metastatic infiltration into the central nervous system.

In some embodiments, the method described herein further comprises administration of one or more other therapeutic agents. In some aspects, the other therapeutic agent aims to treat or prevent cancer. In some embodiments, the other therapeutic is a chemotherapeutic agent. Common chemotherapeutics include, but are not limited to, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, 5-fluorouracil, and the like. In some embodiments, the other therapeutic is an agent used in radiation therapy for the treatment of cancer; indeed, in some embodiments, the method is part of a treatment regimen that includes radiation therapy. Further, the method of the disclosure can be performed in connection with surgical resection of a tumor, such as a glioma (e.g., glioblastoma).

The present disclosure additionally provides kits comprising a nanoparticle composition in containers with instructions for use to evaluate the sensitivity of a subject or tumor to immunotherapy. In some embodiments, the kit comprises one or more components suitable for culturing tumor cells and contacting the tumor cells with the nanoparticles. In some aspects, the kit further comprises other therapeutic or diagnostic agents or pharmaceutically acceptable carriers (e.g., solvents, buffers, diluents, etc.), including any of those described herein.

Aspects of the disclosure include the following:

Aspect 1. A method of identifying a tumor for immunotherapy, the method comprising: a) culturing tumor cells obtained from a subject; b) exposing the tumor cells to nanoparticles comprising a positively-charged surface and an interior comprising (i) a core and (ii) at least two nucleic acid layers, wherein each nucleic acid layer is positioned between a cationic lipid bilayer; and c) measuring interferon-alpha and C-X-C Motif Chemokine Ligand 10 (CXCL10) produced by the tumor cells.

Aspect 2. The method of Aspect 1, wherein the method further comprises measuring interferon-beta.

Aspect 3. The method of Aspect 1 or Aspect 2, wherein the method further comprises measuring interleukin 6 (IL-6).

Aspect 4. The method of any one of Aspects 1-3, wherein the nanoparticles comprise a zeta potential of about 40 mV to about 60 mV.

Aspect 5. The method of Aspect 4, wherein the nanoparticles comprise a zeta potential of about 50 mV.

Aspect 6. The method of any one of Aspects 1-5, wherein the nanoparticles comprise nucleic acid molecules and cationic lipid at a ratio of about 1 to about 5 to about 1 to about 20.

Aspect 7. The method of any one of Aspects 1-6, wherein the cationic lipid is DOTAP or DOTMA.

Aspect 8. The method of any one of Aspects 1-7, wherein the nanoparticles do not comprise a non-cationic lipid.

Aspect 9. The method of any one of Aspects 1-8, wherein the nucleic acid molecules are mRNA molecules.

Aspect 10. The method of any one of Aspects 1-9, wherein the method further comprises d) administering the nanoparticles to the subject.

Aspect 11. The method of any one of Aspects 1-10, wherein the nanoparticle comprises at least four nucleic acid layers, each of which is positioned between a cationic lipid bilayer.

Aspect 12. The method of any one of Aspects 1-11, wherein the outermost layer of the nanoparticle comprises a cationic lipid bilayer.

Aspect 13. The method of any one of Aspects 1-12, wherein the core comprises a cationic lipid bilayer.

Aspect 14. The method of any one of Aspects 1-13, wherein the core comprises less than about 0.5 wt % nucleic acid.

Aspect 15. The method of any one of Aspects 1-14, wherein the nanoparticle comprises a zeta potential of about 45 mV to about 55 mV.

Aspect 16. The method of Aspect 15, wherein the nanoparticle comprises a zeta potential of about 50 mV.

Aspect 17. The method of any one of Aspects 1-16, wherein the immunotherapy is an immune checkpoint inhibitor (ICI).

Aspect 18. The method of any one of Aspects 1-17, wherein the method comprises administering an ICI to the subject.

Aspect 19. The method of Aspects 17 or 18, wherein the ICI is a PD-L1 inhibitor.

Aspect 20. The method of Aspect 19, wherein the PD-L1 inhibitor is a PD-L1 antibody.

Aspect 21. The method of any one of Aspects 1-20, further comprising administering to the subject a population of second nanoparticles comprising a positively-charged surface and an interior comprising (i) a core and (ii) at least two nucleic acid layers, wherein each nucleic acid layer is positioned between a cationic lipid bilayer.

Aspect 22. The method of Aspect 21, wherein the second nanoparticles comprise at least four nucleic acid layers, each of which is positioned between a cationic lipid bilayer.

Aspect 23. The method of Aspects 21 or 22, wherein the second nanoparticles comprise five or more nucleic acid layers, each of which is positioned between a cationic lipid bilayer.

Aspect 24. The method of any one of Aspects 21-23, wherein the outermost layer of the second nanoparticles comprise a cationic lipid bilayer.

Aspect 25. The method of any one of Aspects 21-24, wherein the core of the second nanoparticles comprises a cationic lipid bilayer.

Aspect 26. The method of any one of Aspects 21-25, wherein the core of the second nanoparticles comprises less than about 0.5 wt % nucleic acid.

Aspect 27. The method of any one of Aspects 21-26, wherein the second nanoparticles comprise a zeta potential of about 40 mV to about 60 mV.

Aspect 28. The method of Aspect 27, wherein the second nanoparticles comprise a zeta potential of about 45 mV to about 55 mV.

Aspect 29. The method of Aspect 27, wherein the second nanoparticles comprise a zeta potential of about 50 mV.

Aspect 30. The method of any one of Aspects 21-29, wherein the second nanoparticles comprise nucleic acid molecules and cationic lipid at a ratio of about 1 to about 5 to about 1 to about 20, optionally, about 1 to about 15 or about 1 to about 7.5.

Aspect 31. The method of any one of Aspects 21-30, wherein the cationic lipid is DOTAP or DOTMA.

Aspect 32. The method of any one of Aspects 21-31, wherein the nucleic acid molecules of the second nanoparticles are RNA molecules.

Aspect 33. The method of Aspect 32, wherein the RNA molecules are mRNA.

Aspect 34. The method of Aspects 32 or 33, wherein the second comprise a mixture of RNA molecules.

Aspect 35. The method of Aspect 34, wherein the subject has a tumor and the mixture of RNA is RNA isolated from the tumor of the subject, optionally, wherein the tumor is a malignant brain tumor, optionally, a glioblastoma, medulloblastoma, diffuse intrinsic pontine glioma, or a peripheral tumor with metastatic infiltration into the central nervous system.

Aspect 36. A method of treating a subject with cancer, the method comprising a) culturing tumor cells obtained from the subject; b) exposing the tumor cells to nanoparticles comprising a positively-charged surface and an interior comprising (i) a core and (ii) at least two nucleic acid layers, wherein each nucleic acid layer is positioned between a cationic lipid bilayer; c) measuring interferon-alpha and C-X-C Motif Chemokine Ligand 10 (CXCL10) produced by the tumor cells; and d) administering an immune checkpoint inhibitor to the subject.

Aspect 37. The method of Aspect 36, wherein the method further comprises measuring interferon-beta produced by the tumor cells.

Aspect 38. The method of Aspects 36 or 37, wherein the method further comprises measuring interleukin 6 (IL-6) produced by the tumor cells.

Aspect 39. The method of any of the Aspects above, wherein the method comprises measuring CCL4.

### EXAMPLES

The following examples are given merely to illustrate the present invention and not in any way to limit its scope.

### Example 1

This example describes a method of making nanoparticles of the present disclosure.

### Preparation of DOTAP Liposomes

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask was allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi rotavapor water bath was set to 50° C. PBS (50 mL) was added into the evaporating flask using a 25-mL disposable serological pipette. The evaporating flask was positioned in the Buchi rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50° C. for 1 hour. During the 1 hour incubation, the bottle was vortexed every 10 min. The second 500 mL PBS bottle was allowed to rest on overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 μm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 μm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4° C.

### RNA Preparation

Prior to incorporation into NPs, RNA was prepared in one of a few ways. Total tumor RNA was prepared by isolating total RNA (including rRNA, tRNA, mRNA) from tumor cells. In vitro transcribed mRNA was prepared by carrying out in vitro transcription reactions using cDNA templates produced by reverse transcription of total tumor RNA. Tumor antigen-specific and Non-specific RNAs were either made in-house or purchased from a vendor.

Total Tumor RNA: Total tumor-derived RNA from tumor cells (e.g., B16F0, B16F10, and KR158-luc) is isolated using commercially available RNeasy mini kits (Qiagen) based on manufacturer instructions.

In vitro transcribed mRNA: Briefly, RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

Tumor Antigen-Specific and Non-Specific mRNA: Plasmids comprising DNA encoding tumor antigen-specific RNA (RNA encoding, e.g., pp65, OVA) and non-specific RNA (RNA encoding, e.g., Green Fluorescent Protein (GFP), luciferase) are linearized using restriction enzymes (i.e., SpeI) and purified with Qiagen PCR MiniElute kits. Linearized DNA is subsequently transcribed using the mmRNA in vitro transcription kit (Life technologies, Invitrogen) and cleaned up using RNA Maxi kits (Qiagen). In alternative methods, non-specific RNA is purchased from Trilink Biotechnologies (San Diego, CA).

### Preparation of Multilamellar RNA Nanoparticles (NPs)

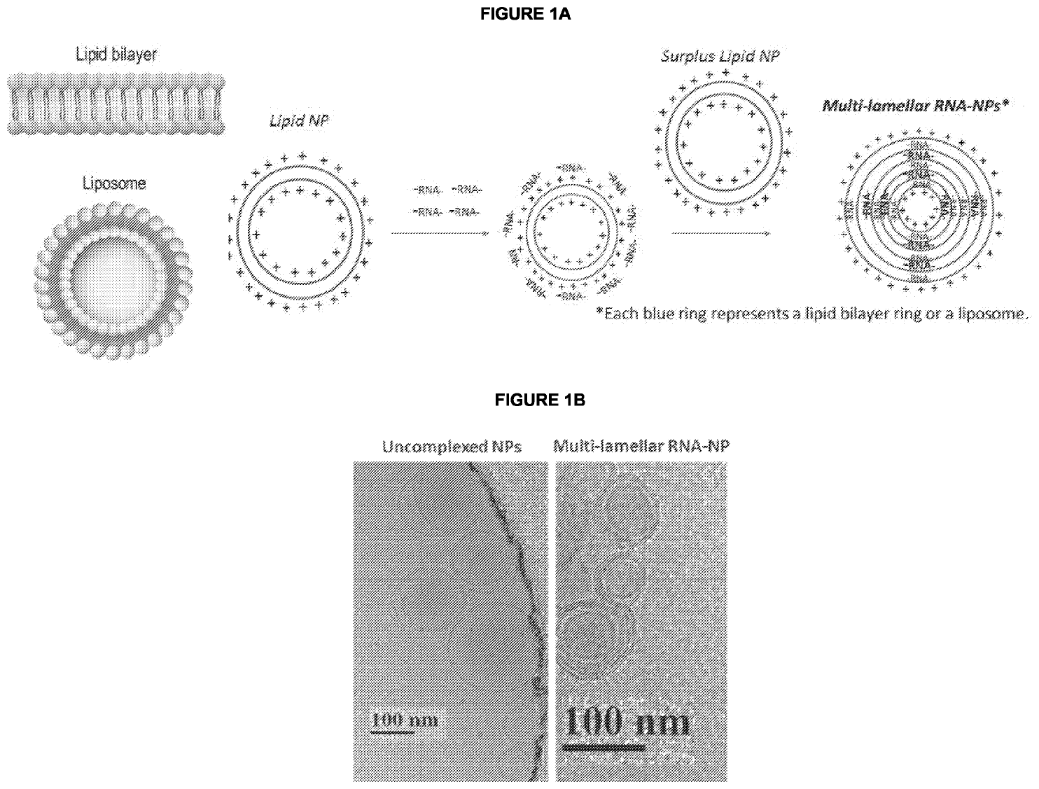

The DOTAP lipid NPs were complexed with RNA to make multilamellar RNA-NPs which were designed to have several layers of mRNA contained inside a tightly coiled liposome with a positively charged surface and an empty core (FIG. 1A). Briefly, in a safety cabinet, RNA was thawed from −80° C. and then placed on ice, and samples comprising PBS and DOTAP (e.g., DOTAP lipid NPs) were brought up to room temperature. Once components were prepared, the desired amount of RNA was mixed with PBS in a sterile tube. To the sterile tube containing the mixture of RNA and PBS, the appropriate amount of DOTAP lipid NPs was added without any physical mixing (without e.g., inversion of the tube, without vortexing, without agitation). The mixture of RNA, PBS, and DOTAP was incubated for about 15 minutes to allow multilamellar RNA-NP formation. After 15 min, the mixture was gently mixed by repeatedly inverting the tube. The mixture was then considered ready for systemic (i.e. intravenous) administration.

The amount of RNA and DOTAP lipid NPs (liposomes) used in the above preparation is pre-determined or pre-selected. In some instances, a ratio of about 15 pg liposomes per about 1 pg RNA were used. For instance, about 75 pg liposomes are used per ˜5 pg RNA or about 375 pg liposomes are used per ˜25 pg RNA. In other instances, about 7.5 pg liposomes were used per 1 pg RNA. Thus, in exemplary instances, about 1 μg to about 20 pg liposomes are used for every pg RNA used.

### Example 2

This example describes the characterization of the nanoparticles of the present disclosure.

### Cryo-Electron Microscopy (CEM)

CEM was used to analyze the structure of multilamellar RNA-NPs prepared as described in Example 1 and control NPs devoid of RNA (uncomplexed NPs) which were made by following all the steps of Example 1, except for the steps under “RNA Preparation” and “Preparation of Multilamellar RNA nanoparticles (NPs)”. CEM was carried out as essentially described in Sayour et al., Nano Lett 17(3) 1326-1335 (2016). Briefly, samples comprising multilamellar RNA-NPs or control NPs were kept on ice prior to being loaded in a snap-freezed in Vitrobot (and automated plunge-freezer for cryoTEM, that freezes samples without ice crystal formation, by controlling temperature, relative humidity, blotting conditions and freezing velocity). Samples were then imaged in a Tecnai G2 F20 TWIN 200 kV/FEG transmission electron microscope with a Gatan UltraScan 4000 (4 k×4 k) CCD camera. The resulting CEM images are shown in FIG. 1B. The right panel is a CEM image of multilamellar RNA-NPs and the left panel is a CEM image of control NPs (uncomplexed NPs). As shown in FIG. 1B, the control NPs contained at most 2 layers, whereas multilamellar RNA NPs contained several layers.

### Zeta Potentials

Zeta potentials of multilamellar RNA NPs were measured by phase analysis light scattering (PALS) using a Brookhaven ZetaPlus instrument (Brookhaven Instruments Corporation, Holtsville, NY), as essentially described in Sayour et al., Nano Lett 17(3) 1326-1335 (2016). Briefly, uncomplexed NPs or RNA-NPs (200 μL) were resuspended in PBS (1.2 mL) and loaded in the instrument. The samples were run at 5 runs per sample, 25 cycles each run, and using the Smoluchowski model.

The zeta potential of the multilamellar RNA NPs prepared as described in Example 1 was measured at about +50 mV. Interestingly, this zeta potential of the multilamellar RNA NPs was much higher than those described in Sayour et al., Oncoimmunology 6(1): e1256527 (2016), which measured at around +27 mV. Without being bound to any particular theory, the way in which the DOTAP lipid NPs are made for use in making the multilamellar RNA NPs (Example 1) involving a vacuum-seal method for evaporating off chloroform leads to less environmental oxidation of the DOTAP lipid NPs, which, in turn, may allow for a greater amount of RNA to complex with the DOTAP NPs and/or greater incorporation of RNA into the DOTAP lipid NPs.

### RNA Incorporation by Gel Electrophoresis:

A gel electrophoresis experiment was conducted to measure the amount of RNA incorporated into ML liposomes. Based on this experiment, it was qualitatively shown that nearly all, if not all, of the RNA used in the procedure described in Example 1 was incorporated into the DOTAP lipid NPs. Additional experiments to characterize the extent of RNA incorporation are carried out by measuring RNA-NP density and comparing this parameter to that of lipoplexes.

### Example 3

This example describes a comparison of the nanoparticles of the present disclosure to cationic RNA lipoplexes and anionic RNA lipoplexes.

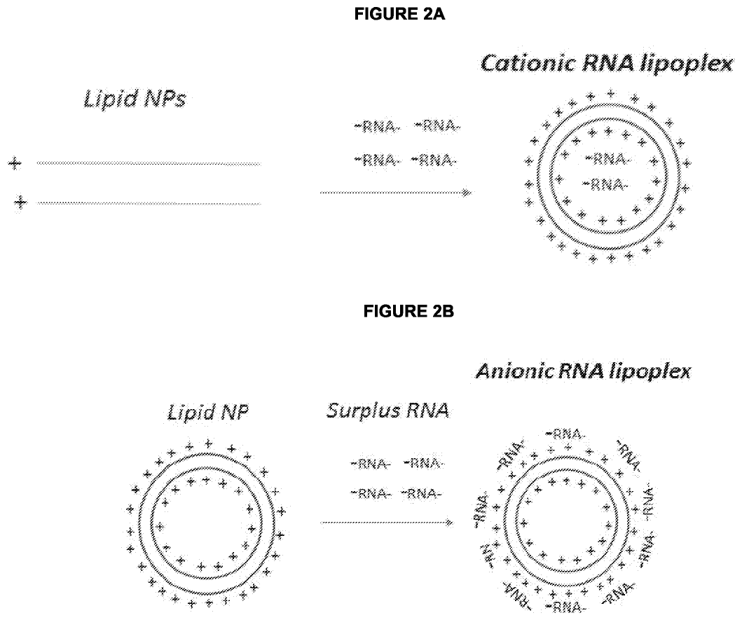

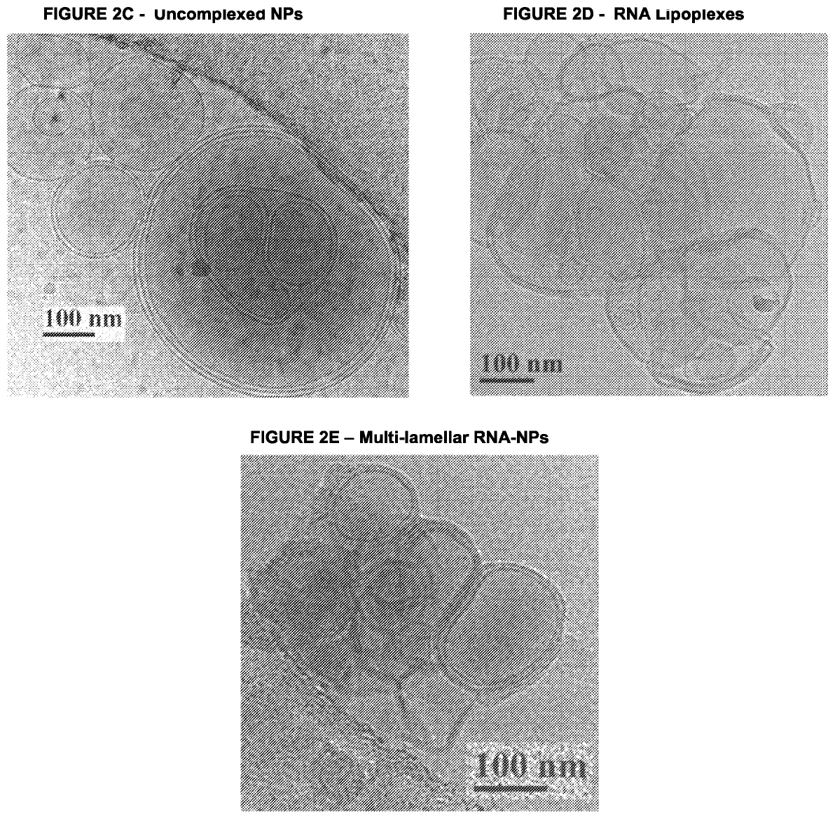

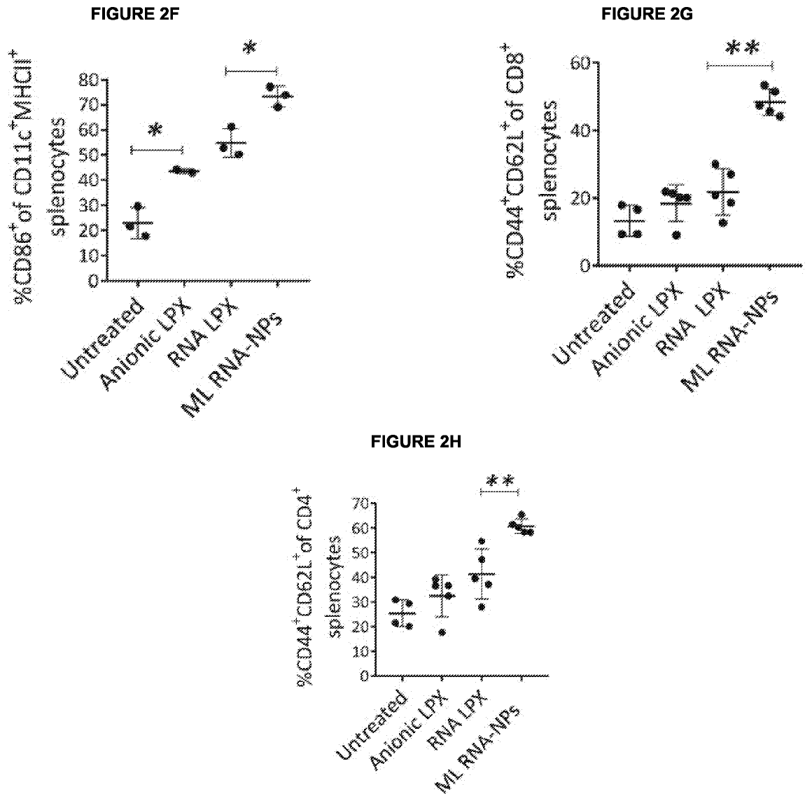

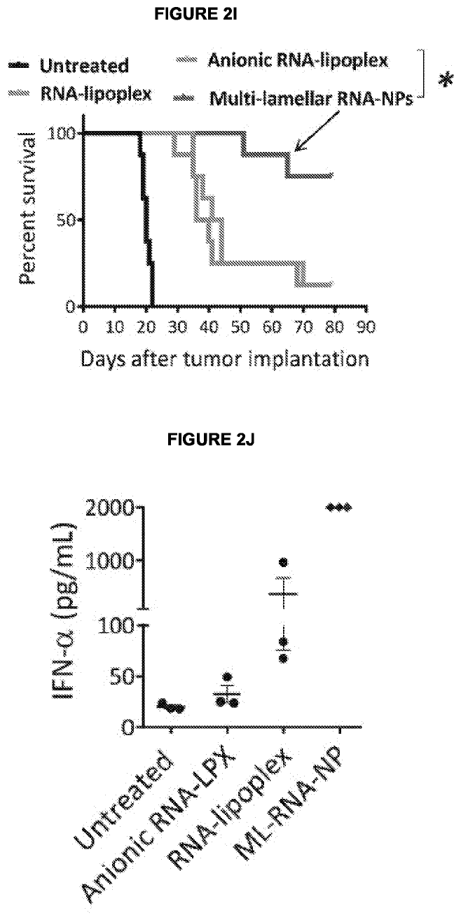

Cationic lipoplexes (LPX) were first developed with mRNA in the lipid core shielded by a net positive charge located on the outer surface (FIG. 2A). Anionic RNA lipoplexes (FIG. 2B) have been developed with an excess of RNA tethered to the surface of bi-lamellar liposomes. RNA-LPX were made by mixing RNA and lipid NP at ratios to equalize charge. Anionic RNA-NPs were made by mixing RNA and lipid NP at ratios to oversaturate lipid NPs with negative charge. Various aspects of the RNA-LPX and anionic RNA LPX were then compared to the multilamellar RNA NPs described in the above examples.

Cryo-Electron Microscopy (CEM) was used to compare the structures of the RNA LPX and the multilamellar RNA-NPs prepared as described in Example 1. Uncomplexed NPs were used as a control. CEM was carried out as essentially described in Example 2. FIG. 2C is a CEM image of uncomplexed NPs, FIG. 2D is a CEM image of RNA LPXs (wherein that mass ratio of liposome to RNA is 3.75:1) and FIG. 2E is a CEM image of the multilamellar RNA-NPs (wherein that mass ratio of liposome to RNA is 15:1). These data support that more RNA is held by the ML RNA-NPs. Additional data show that the concentration drops more with ML RNA-NP complexation versus RNA LPX supporting multilamellar formation of ML RNA-NPs not observed by simple mixing of equivalent amounts of RNA and lipid NPs by mass or charge (i.e. RNA-LPX and anionic RNA-LPX respectively). This supports that more RNA is “held” by ML RNA-NPs described herein.

Also, an experiment was conducted to determine where the anionic LPXs localize upon administration to mice. Anionic LPXs localized to the spleens of animals upon administration.

RNA LPX, anionic lipoplex (LPX) or multilamellar RNA-NPs were administered to mice and spleens were harvested one week later for assessment of activated DCs (*p<0.05 unpaired t test). The RNA used in this experiment was tumor-derived mRNA from the K7M2 tumor osteosarcoma cell line. As shown in FIG. 2F, mice treated with multilamellar RNA NPs exhibited the highest levels of activated DCs.

Anionic tumor mRNA-lipoplexes, tumor mRNA-lipoplexes, and multilamellar tumor mRNA loaded NPs were compared in a therapeutic lung cancer model (K7M2) (n=5-8/group). Each vaccine was intravenously administered weekly (×3) (**p<0.01, Mann Whitney). The % CD44+CD62L+ of CD8+ splenocytes is shown in FIG. 2G and the % CD44+CD62L+ of CD4+ splenocytes is shown in FIG. 2H. Also, FIG. 2J shows that multilamellar (ML) RNA-NPs mediate substantially increased IFN-alpha, which is an innate anti-viral cytokine. This demonstrates that ML RNA-NPs allow for substantially greater innate immunity which is enough to drive efficacy from even non-antigen specific ML RNA-NPs. These data also indirectly support that ML RNA-NPs increase the number of activated plasmacytoid dendritic cells (pDCs) which cells are the most important producers of IFN-alpha. Taken together, the data demonstrates the superior efficacy of multilamellar tumor specific RNA-NPs, relative to anionic LPX and RNA LPX.

Anionic tumor mRNA-lipoplexes, cationic tumor mRNA-lipoplexes and multilamellar tumor mRNA loaded NPs were compared in a therapeutic lung cancer model (K7M2) (n=8/group). Each vaccine was iv administered weekly (×3), *p<0.05, Gehan Breslow-Wilcoxon test. The percent survival was measured by Kaplan-Meier Curve analysis. As shown in FIG. 2I, multilamellar tumor specific RNA-NPs mediated superior efficacy, compared to cationic RNA lipoplexes and anionic RNA lipoplexes, for increasing survival.

The ability of multilamellar RNA-NP to activate the innate immune response in vivo also was examined in the glioma tumor microenvironment.

RNA-NPs localize to perivascular regions of tumors and reprogram the TME in favor of activated myeloid cells. K-luc bearing animals (n=5/group) were vaccinated with tumor RNA-NPs or NPs alone. Tumors were harvested 48 h later for RNA-seq analysis. In animals receiving RNA-NPs, a significant upregulation of gene signatures for BATF3, IRFs, and IFN response genes was observed. In particular, the RNA-NP of the invention significantly upregulated expression of BATF3 (associated with effector dendritic cell phenotype), IRF5 and IRF7 (interferon regulatory factors), and ISG15 and IFITM3 (interferon response genes). These genes have been shown to be essential for sensitizing immunotherapeutic responses. As such, the RNA-NPs upregulate critical innate immune gene signatures in the glioma tumor microenvironment that associated with effector immune response, in effect turning tumors from “cold” to “hot,” allowing immune checkpoint inhibitors to be active where they were previously ineffective prior to RNA-NP treatment.

Herein it is demonstrated that the multilamellar RNA-NP formulation targeting physiologically relevant tumor antigens is more immunogenic (FIGS. 2F-2H, 2J) and significantly more efficacious (FIG. 2I) compared with anionic LPX and RNA LPX. Without being bound to any particular theory, by altering RNA-lipid ratios and increasing the zeta potential, a novel RNA-NP design composed of multi-lamellar rings of tightly coiled mRNA has been developed (FIG. 1C), which multi-lamellar design is thought to facilitate increased NP uptake of mRNA (condensed by alternating positive/negative charge) for enhanced particle immunogenicity and widespread in vivo localization to the periphery and tumor microenvironment (TME). Systemic administration of these multi-lamellar RNA-NPs localize to lymph nodes, reticuloendothelial organs (i.e. spleen and liver) and to the TME, activating DCs therein (based on increased expression of the activation marker CD86 on CD11c+ cells). These activated DCs prime antigen specific T cell responses, which lead to anti-tumor efficacy (with increased TILs) in several tumor models.

### Example 4

Innate cytokines including INF-α and INF-β are produced early on in the setting of tumorigenesis from checkpoint sensitive tumors in response to nucleic acid (DNA, RNA) release. Their production elucidates the development and regulation of the immune systems innate response. Unfortunately, there is no assay for prospectively knowing whether a given patient will respond to immune checkpoint inhibitors (ICIs).

The study described below was based, at least in part, on the hypothesis that murine cell lines known to respond ICIs would have a proinflammatory cytokine release signature under viremic stress compared with known non-responsive murine tumor cells. Viremia was simulated in vitro against cancer cell lines Glioma 261, KR158b-Luc, B16F0, and B16F10 OVA cells using the nanoparticles of the disclosure loaded with green fluorescent protein-encoding mRNA as a viral mimic. Through a Mouse Procarta IFN 2-Plex Array, the amount of both INF-α and INF-β released by each cell line was measured. In addition, using a mouse cytokine array/chemokine array-31 plex, additional chemokine expression was measured for each cell line. Our results indicated the highest INF-α and INF-β release were found in GL261 cells treated with RNA-loaded nanoparticles compared to other cell lines, suggesting these to be the most immunogenic. Interestingly, this cell line is most responsive to immune checkpoint inhibitors with long-term survival benefit. In addition, significant Interleukin-6 and CXCL10 expression was recorded in GL-26 and B16F10 Ova cells transfected with RNA-nanoparticles. The intensity of green fluorescent protein also was assessed using flow cytometry. The highest expression was observed in RNA-nanoparticle-treated GL261 cells. These results allow for creation of a model system to quickly assess whether a particular malignancy is immunogenic, allowing for informed treatment with checkpoint inhibitors or lack thereof in poorly immunogenic contexts.

### Methods

Cell lines: Mouse tumor cell lines, GL-261, KR158b-Luc, B16F10 Ova, and B16F0 were thawed and cultured in Dulbecco® Modified Eagle® Medium with 10% fetal bovine serum and 1% penicillin/streptomycin. Cells were incubated in T75 flasks at 37° with 5% C02. Cells were passaged every 3 to 4 days and observed for contamination.

Cell transfection: Upon confluency, cells were counted using a hemocytometer and the treatment volumes were determined. Cells were seeded in 6 well plates and transfected with treatment groups RNA+ nanoparticles, nanoparticles, and untreated. Cells treated with RNA-nanoparticles received a combination mRNA and DOTAP while nanoparticle treated cells received no RNA.

Supernatant collection: Supernatant was collected approximately 24 h after transfection and frozen. After an additional 48 h, supernatant was thawed and prepared for shipping by combination with pbs for a mouse Procarta IFN 2-Plex Array and a mouse cytokine array/chemokine array-31 plex assessed by Eve Technologies.

### Results

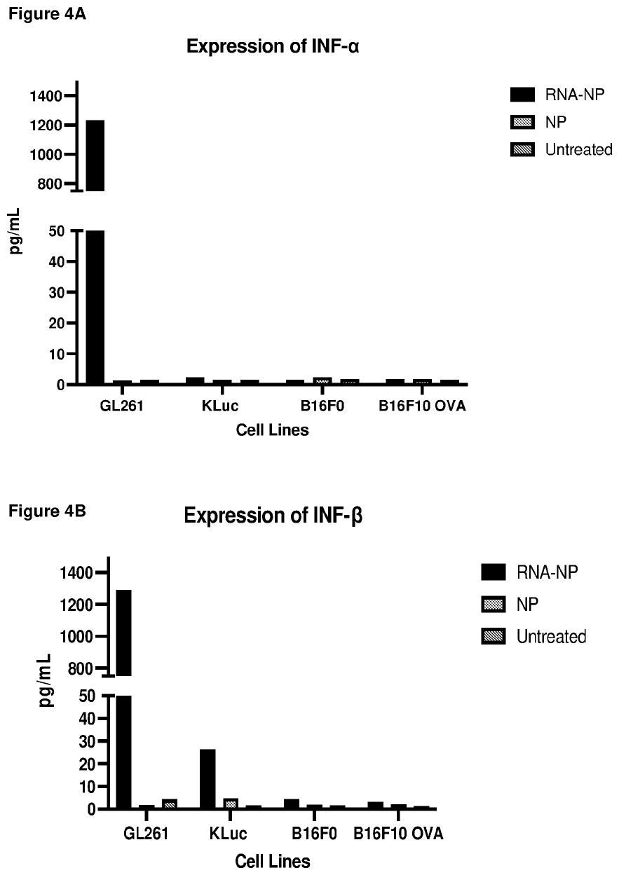

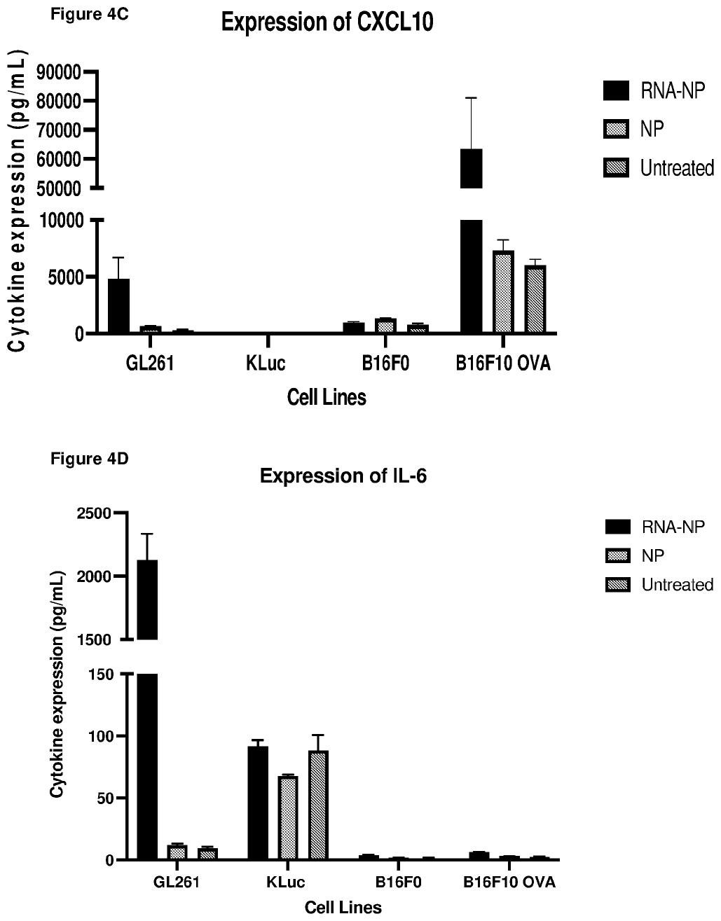

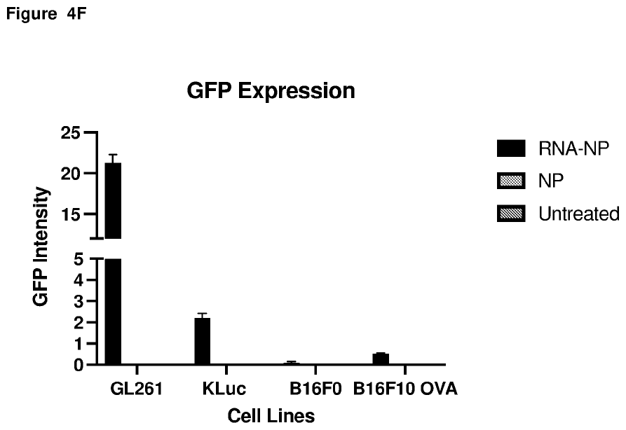

Cytokine, chemokine, and GFP expression in transfected cells is illustrated in FIGS. 4A-E. These data demonstrate that immunogenic tumors (i.e., tumors responsive to immune checkpoint inhibitors) have distinct cytokine signature based on release and magnitude of type I interferon release and release of CXCL10. These results do not appear to completely correlate with percent transfection, suggesting that tumor cell immunogenicity is governed by unique tumor biology that could be predicted (i.e., checkpoint responder versus nonresponder) based on challenge assay with RNA-NPs.

These results indicate that cytokine signatures are significantly different under immunogenic stress from ICI responsive versus non-responsive murine tumors. ICI responsive tumors (GL261) release significantly greater INF-α and INF-β following RNA-loaded nanoparticles, suggesting these (as expected) to be the most immunogenic tumors. RNA-loaded nanoparticles also induced chemokine expression in B16F10 OVA cells. These findings allow for use of the nanoparticles of the disclosure to quickly assess whether a particular malignancy is immunogenic, allowing for informed treatment with checkpoint inhibitors or alternative treatment strategies in poorly immunogenic settings.

### Example 5

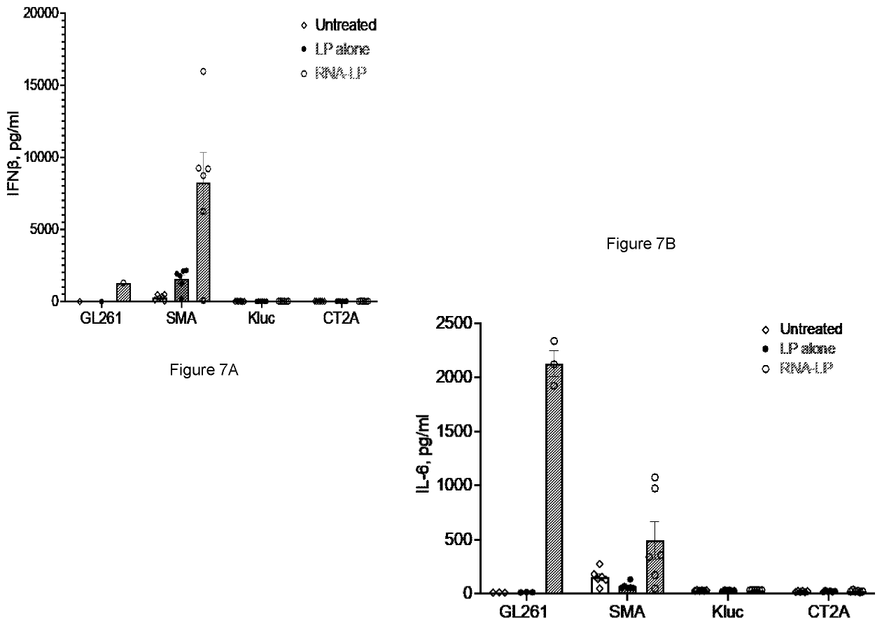

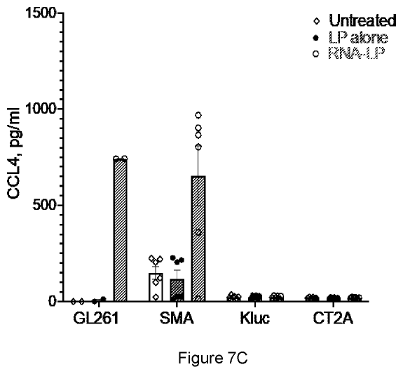

To prospectively determine whether individual brain tumors respond to ICIs, the RNA nanoparticles described herein were utilized to determine cancer cell immunogenicity. These particles activate intracellular pathogen recognition receptors but can also stimulate endosomal toll-like receptors, providing a single approach to simultaneously activate multiple pathogen recognition receptors (PRRs) and determine the ability of a stressed cell to elicit an innate response. Murine brain tumor lines known to respond or resist treatment following immune checkpoint inhibitors (ICIs) were challenged with these nanoparticles. Downstream production of proinflammatory cytokines were analyzed as predictors of ICI response. Brain tumor lines GL261 and SMA-560 were selected as ICI responsive, and KR158b-luc and CT-2A tumor lines were selected as ICI unresponsive. These tumor cells lines were cultured in 2D in vitro and transfected with GFP mRNA. While transfection rates did not appear to substantially change across cell lines, there were marked differences in cytokine response signatures across the brain tumor cell lines. Following mRNA challenge, ICI responsive tumors GL261 and SMA-500 showed increases in pro-inflammatory cytokines IFN-β (interferon-beta), IL-6 and in CCL4 chemokine associated with dendritic cell/T cell trafficking. See FIGS. 7A-7C. ICI responsive tumors (GL261, SMA-500) released significantly greater INF-β, IL-6, and CCL4 following RNA-loaded lipid-particles exposure, suggesting these (as expected) to be the most immunogenic tumors.

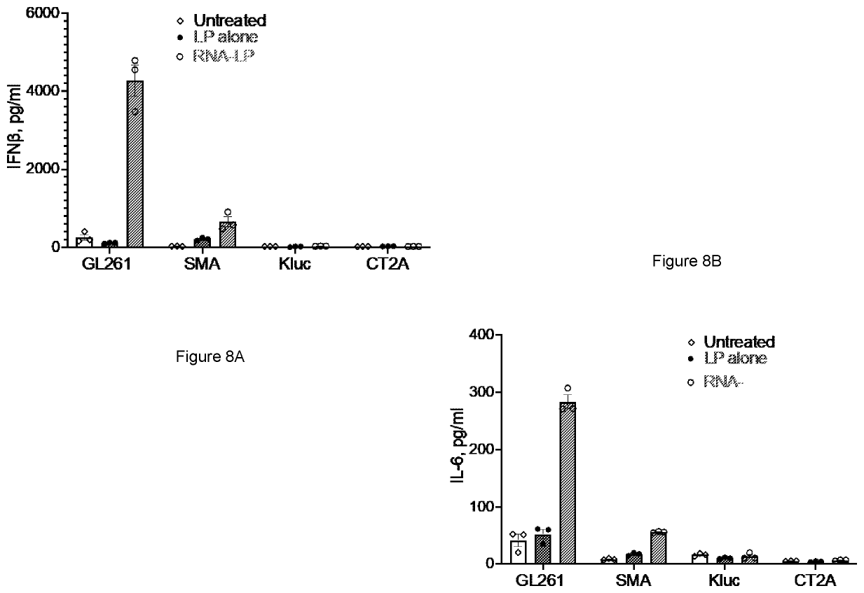

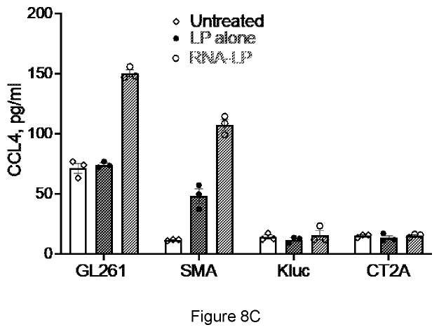

Three-dimensional modeling of patient derived tumors for mRNA challenge was performed. Canines with primary gliomas were enrolled onto clinical trials and began growing tumors in 3D using liquid-like solid technology that rapidly allows for simultaneous growth of 96 patient-derived microtumors. Bhattacharjee, T. et al. Writing in the granular gel medium. Sci Adv 1, e1500655, doi:10.1126/sciadv.1500655 (2015). This system demonstrated that canine glioma recapitulates features of human glioblastoma based on infiltrative pattern and finger-like projections. In separate studies, 3D human GBM tumoroids were perfused with RNA nanoparticles as evidenced by GFP expression, demonstrating the ability to set up real-time, patient derived explants for mRNA challenge to predict immunogenicity. These data validate the ability to grow personalized tumoroids for mRNA challenge. To demonstrate that mRNA perfusion of 3D tumoroids elicits similar cytokine response observed with 2D culture of cell-lines, GL261 and KR158b-luc 3D tumoroids were challenged with GFP mRNA, which elicited the same increases in IFN-β, IL-6, and CCL4 in GL261 and SMA-500 (see FIGS. 8A-8C), but not in KR158b-luc or CT-2A. Thus, 3D modeling of murine gliomas confirmed cytokine response signature unique to checkpoint responsive malignancies.

Cancer cells can evolve to subvert immune recognition while mediating recruitment of myeloid derived suppressor cells and tumor associated macrophages that exclude T cells from the tumor microenvironment (TME). Alternatively, immunogenic tumors can alert DCs and T cells through innate production of cytokines/chemokines eliciting adaptive immunity that is stymied through expression of immune checkpoints. These innate cytokines (e.g., IFN-alpha) are produced early on in the setting of tumorigenesis from checkpoint sensitive tumors in response to nucleic acid (DNA, RNA) release, elucidating the development and regulation of our immune systems innate response. Rapid cell division creates competition and stress among cancer cell subpopulations, leading to apoptosis and release of damage associated molecular patterns (DAMPs) and pathogen associated molecular patterns (PAMPs, free RNA, DNA). These PAMPs can trigger PRRs in the tumor microenvironment and induction of type I interferon responses that recruit DCs/T cells predisposing cancer immunogenicity and exhaustion. Alternatively, tumor cells may evolve mechanisms or grow from stem progenitor states that lack PRR machinery that stymie this process to prevent immune recruitment. These ‘hot’ versus ‘cold’ tumors are difficult to rapidly predict. By encapsulating single/double-stranded stranded elements and eliciting DAMPs through cationic charge, a multilamellar RNA-LP approach was developed to rapidly predict cancer cell immunogenicity through induction of type I interferon responses as a surrogate for ICI responsiveness. The results described herein indicate that cytokine signatures are significantly different under immunogenic stress from mRNA challenge in ICI responsive versus non-responsive tumors. The method described herein allows quick assessment of how tumor cells respond to these triggers, which provides a way to prospectively manage patients (i.e., allowing for informed treatment with checkpoint inhibitors or lack thereof in poorly immunogenic settings).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. The disclosure contemplates embodiments described as “comprising” a feature to include embodiments which “consist of” or “consist essentially of” the feature. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 10%, up to 5%, or up to 1% of a given value.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. In any of the ranges described herein, the endpoints of the range are included in the range. However, the description also contemplates the same ranges in which the lower and/or the higher endpoint is excluded.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

What is claimed:

1. A method of identifying a tumor for immunotherapy, the method comprising:
a) culturing tumor cells obtained from a subject;
b) exposing the tumor cells to nanoparticles comprising a positively-charged surface and an interior comprising (i) a core and (ii) at least two nucleic acid layers, wherein each nucleic acid layer is positioned between a cationic lipid bilayer; and
c) measuring interferon-alpha and interleukin 6 (IL-6) produced by the tumor cells.

2. The method of claim 1, wherein the method further comprises measuring interferon-beta.

3. The method of claim 1 or claim 2, wherein the method further comprises measuring C-X-C Motif Chemokine Ligand 10 (CXCL10).

4. The method of any one of claims 1-3, wherein the nanoparticles comprise a zeta potential of about 40 mV to about 60 mV.

5. The method of claim 4, wherein the nanoparticles comprise a zeta potential of about 50 mV.

6. The method of any one of claims 1-5, wherein the nanoparticles comprise nucleic acid molecules and cationic lipid at a ratio of about 1 to about 5 to about 1 to about 20.

7. The method of any one of claims 1-6, wherein the cationic lipid is DOTAP or DOTMA.

8. The method of any one of claims 1-7, wherein the nanoparticles do not comprise a non-cationic lipid.

9. The method of any one of claims 1-8, wherein the nucleic acid molecules are mRNA molecules.

10. The method of any one of claims 1-9, wherein the method further comprises d) administering the nanoparticles to the subject.

11. The method of any one of claims 1-10, wherein the nanoparticle comprises at least four nucleic acid layers, each of which is positioned between a cationic lipid bilayer.

12. The method of any one of claims 1-11, wherein the outermost layer of the nanoparticle comprises a cationic lipid bilayer.

13. The method of any one of claims 1-12, wherein the core comprises a cationic lipid bilayer.

14. The method of any one of claims 1-13, wherein the core comprises less than about 0.5 wt % nucleic acid.

15. The method of any one of claims 1-14, wherein the nanoparticle comprises a zeta potential of about 45 mV to about 55 mV.

16. The method of claim 15, wherein the nanoparticle comprises a zeta potential of about 50 mV.

17. The method of any one of claims 1-16, wherein the immunotherapy is an immune checkpoint inhibitor (ICI).

18. The method of any one of claims 1-17, wherein the method comprises administering an ICI to the subject.

19. The method of claim 17 or claim 18, wherein the ICI is a PD-L1 inhibitor.

20. The method of claim 19, wherein the PD-L1 inhibitor is a PD-L1 antibody.

21. The method of any one of claims 1-20, further comprising administering to the subject a population of second nanoparticles comprising a positively-charged surface and an interior comprising (i) a core and (ii) at least two nucleic acid layers, wherein each nucleic acid layer is positioned between a cationic lipid bilayer.

22. The method of claim 21, wherein the second nanoparticles comprise at least four nucleic acid layers, each of which is positioned between a cationic lipid bilayer.

23. The method of claim 21 or 22, wherein the second nanoparticles comprise five or more nucleic acid layers, each of which is positioned between a cationic lipid bilayer.

24. The method of any one of claims 21-23, wherein the outermost layer of the second nanoparticles comprise a cationic lipid bilayer.

25. The method of any one of claims 21-24, wherein the core of the second nanoparticles comprises a cationic lipid bilayer.

26. The method of any one of claims 21-25, wherein the core of the second nanoparticles comprises less than about 0.5 wt % nucleic acid.

27. The method of any one of claims 21-26, wherein the second nanoparticles comprise a zeta potential of about 40 mV to about 60 mV.

28. The method of claim 27, wherein the second nanoparticles comprise a zeta potential of about 45 mV to about 55 mV.

29. The method of claim 27, wherein the second nanoparticles comprise a zeta potential of about 50 mV.

30. The method of any one of claims 21-29, wherein the second nanoparticles comprise nucleic acid molecules and cationic lipid at a ratio of about 1 to about 5 to about 1 to about 20, optionally, about 1 to about 15 or about 1 to about 7.5.

31. The method of any one of claims 21-30, wherein the cationic lipid is DOTAP or DOTMA.

32. The method of any one of claims 21-31, wherein the nucleic acid molecules of the second nanoparticles are RNA molecules.

33. The method of claim 32, wherein the RNA molecules are mRNA.

34. The method of claim 32 or claim 33, wherein the second comprise a mixture of RNA molecules.

35. The method of claim 34, wherein the subject has a tumor and the mixture of RNA is RNA isolated from the tumor of the subject, optionally, wherein the tumor is a malignant brain tumor, optionally, a glioblastoma, medulloblastoma, diffuse intrinsic pontine glioma, or a peripheral tumor with metastatic infiltration into the central nervous system.

36. The method of any one of claims 1-35, wherein the method further comprises measuring Chemokine (C-C motif) ligands 4 (CCL4).

37. A method of treating a subject with cancer, the method comprising
a) culturing tumor cells obtained from the subject;
b) exposing the tumor cells to nanoparticles comprising a positively-charged surface and an interior comprising (i) a core and (ii) at least two nucleic acid layers, wherein each nucleic acid layer is positioned between a cationic lipid bilayer;
c) measuring interferon-alpha and interleukin 6 (IL-6) produced by the tumor cells; and
d) administering an immune checkpoint inhibitor to the subject.

38. The method of claim 37, wherein the method further comprises measuring interferon-beta produced by the tumor cells.

39. The method of claim 37 or claim 38, wherein the method further comprises measuring C-X-C Motif Chemokine Ligand 10 (CXCL10) produced by the tumor cells.

40. The method of any one of claims 37-39, wherein the method further comprises measuring Chemokine (C-C motif) ligands 4 (CCL4) produced by the tumor cells.
